(19) United States
(12) Patent Application Publication
(10) Pub. No.: US 2018/0328919 A1
(43) Pub. Date: Nov. 15, 2018

(54) TARGET MODIFICATION FOR TRACKING AND DETECTION

(71) Applicant: Two Pore Guys, Inc., Santa Cruz, CA (US)

(72) Inventors: Trevor J. Morin, Santa Cruz, CA (US); Daniel A. Heller, Santa Cruz, CA (US); William B. Dunbar, Santa Cruz, CA (US); Tyler Shropshire, Santa Cruz, CA (US)

(21) Appl. No.: 15/775,783

(22) PCT Filed: Nov. 23, 2016
(86) PCT No.: PCT/US16/63597

§ 371 Date: May 11, 2018

Related U.S. Application Data
(60) Provisional application No. 62/259,012, filed on Nov. 23, 2015.

(51) Int. Cl. G01N 33/53; G01N 33/94; G01N 33/487; B82Y 15/00; G01N 27/447; G01N 30/96

(52) U.S. Cl. CPC G01N 33/5308; G01N 33/9453; G01N 33/48721; G01N 2030/8827; G01N 27/447; G01N 30/96; B82Y 15/00

(57) ABSTRACT

The present invention is directed to a target molecule modified to facilitate detection in a nanopore deice. The present invention further relates to a method of detecting such a modified target molecule using a nanopore device. It also disclose a method of using such a modified target molecule for tracking and verification of pharmaceutical, chemical or biological products and for measuring various conditions of a sample comprising the modified target molecule.

### 1. CROSS-REFERENCE

This application claims the benefit of U.S. Provisional Application No. 62/259,012 filed on Nov. 23, 2015, which application is incorporated by reference in their entirety.

### 2. BACKGROUND

Detection of nano-scale and micro-scale molecules, such as biological therapeutics, small molecule drugs, ingredients of chemical or biological products, or chemical or biological indicators have great values.

For example, such detection allows tracking and verification of pharmaceutical, chemical or biological products from their point of production through their distributors, and to consumers. Such tracking and verification has been extremely difficult and time consuming. While methods do exist to help track such products and determine the constituents of a particular product at a given time, the standard method is to send the sample to a lab which is often a time consuming process, if even feasible.

Detection of nano-scale and micro-scale molecules also allows measurement of various physical or physiological conditions of samples. For example, there are a number of nano-scale and micro-scale molecules that are known to change their conformation, structure, or stability in response to various conditions, such as pH, toxic compositions, chemical compositions, temperature, etc. However, it has been difficult and time-consuming to detect the physical status of such molecules and obtain relevant information.

Detection of nano-scale and micro-scale molecules using a nanopore device became possible in recent years. Nanopore detection is a quick, portable, cheap, and accurate means of detecting and analyzing nano-scale and micro-scale molecules. However, application of the nanopore detection has been limited to a small number of target molecules, because nanopore detection requires the target molecules to have specific physical properties, (e.g., size, charge, etc.) that allows the molecules to be captured and pass through a nanopore and be detectable by nanopore sensors. Therefore, there has been a need for ways of applying the nanopore technologies to various target molecules, not inherently having the properties.

### 3. SUMMARY

Various aspects disclosed herein can fulfill one or more of the above-mentioned needs. The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved systems and methods.

In some embodiments, the present disclosure provides target molecules modified to facilitate detection in a nanopore device. In some embodiments, the target molecule comprises a first attachment site, wherein said attachment site is bound to a driver molecule. In some embodiments, the target molecule further comprises a second attachment site, wherein said second attachment site is bound to a first payload molecule.

In some embodiments, the present invention provides a complex designed for nanopore detection, comprising: a target molecule comprising a first attachment site; and a driver molecule comprising a first attachment site, wherein the first attachment site of the driver molecule makes a covalent bond to the first attachment site of the target molecule.

In some embodiments, the present invention provides a complex designed for nanopore detection, comprising: a target molecule comprising a first attachment site; and a linker molecule comprising a first attachment site and a second attachment site, wherein the first attachment site of the linker molecule makes a covalent bond to the first attachment site of the target molecule, and the second attachment site of the linker molecule is a binding site for a driver molecule. In some embodiments, the complex further comprises a driver molecule, wherein the driver molecule is bound to the second attachment site of the linker molecule via a covalent bond, an intermediate linker, a Van der Waals bond, an electrostatic bond, a hydrophobic interaction, a pi-stacking interaction, an ionic bond, or another non-covalent electrostatic interaction.

In some embodiments, the linker molecule comprises a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid (PNA), a polyethylene glycol (PEG), a dendrimer, a synthetic polymer, or any combination thereof. In some embodiments, the linker molecule comprises a peptide, a valine-citrulline linker, a maleimidocaproyl (mc) linker, an [N-maleimidomethyl]cyclohexane-1-carboxylate (MCC) linker, a succinimidyl 4-[N-maleimidomethyl]cyclohexane-1-carboxylate (SMCC) linker, a PEG-based linker, a hydrazone linker, an N-succinimidyl-4-(2-pyridyldithio) butanoate (SPDB) linker, or a carbonate linker, or any other homobifunctional or heterobifunctional molecule.

In some embodiments, the target molecule comprises a biological therapeutic. In some embodiments, the biological therapeutic is a monoclonal antibody, a protein, a protease, a peptide, a sugar, a nucleic acid, or any combination thereof.

In some embodiments, the target molecule comprises a small molecule. In some embodiments, the small molecule is a small molecule therapeutic or small molecule diagnostic drug. In some embodiments, the small molecule is a small molecule therapeutic drug selected from the group consisting of an enzyme inhibitor or activator, a receptor antagonist or agonist, and a small molecule modifier of a cellular process or pathway. In some embodiments, the small molecule therapeutic drug is Lisinopril.

In some embodiments, the target molecule comprises an ingredient of a chemical or biological product, selected from the group consisting of a fertilizer, a construction or building material, and a dietary composition. In some embodiments, the ingredient is a nitrogenase. In some embodiments, the ingredient is boronic acid.

In some embodiments, the target molecule is an indicator selected from the group consisting of pH indicator, a toxin indicator, a chemical indicator, a pollutant indicator, or a temperature indicator.

In some embodiments, the driver molecule is a single stranded deoxyribonucleic acid (ssDNA), a double stranded deoxyribonucleic acid (dsDNA), or a ribonucleic acid (RNA). In some embodiments, the driver molecule comprises more than 20 nucleotides.

In some embodiments, the driver molecule binds to the linker molecule via a covalent bond, an intermediate linker, a Van der Waals bond, an electrostatic bond, a hydrophobic interaction, a pi-stacking interaction, an ionic bond, or another non-covalent electrostatic interaction.

In some embodiments, the target molecule further comprises a second attachment site, wherein said second attachment site is bound to a first payload molecule. In some embodiments, the first payload molecule is bound to the target molecule via a covalent bond, an intermediate linker, a Van der Waals bond, an electrostatic bond, a hydrophobic interaction, a pi-stacking interaction, an ionic bond, or another non-covalent electrostatic interaction.

In some embodiments, the driver molecule further comprises a second attachment site, wherein said second attachment site is bound to a second payload molecule. In some embodiments, the second payload molecule is bound to the driver molecule via a covalent bond, an intermediate linker, a Van der Waals bond, an electrostatic bond, a hydrophobic interaction, a pi-stacking interaction, an ionic bond, or another non-covalent electrostatic interaction.

In some embodiments, the first or the second payload molecule is a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid, a polyethylene glycol (PEG), a dendrimer, or any combination thereof.

In some embodiments, the target molecule is bound to a plurality of payload molecules. In some embodiments, the driver molecule is bound to a plurality of payload molecules.

In some embodiments, the first attachment site of the target molecule comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol. In some embodiments, the first attachment site of the driver molecule comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

The present invention further provides a method of detecting a target molecule. In some embodiment, the method comprises the steps of (a) loading a sample suspected to comprise the complex comprising a target molecule in a nanopore device comprising at least one nanopore; (b) applying a voltage across the at least one nanopore; and (c) measuring an electrical signal through the pore that correlates with the presence or absence of the complex in the sample.

In some embodiments, the sample comprises a pharmaceutical composition. In some embodiments, the sample comprises a blood, urine, saliva, or tissue sample from a patient. In some embodiments, the sample comprises water, soil, air, sludge, petroleum, or a chemical or biological product.

In some embodiments, the complex is bound to one or more payload molecules, and wherein the electrical signal further correlates with the binding of said one or more payload molecules.

In some embodiments, the method further comprises a step of determining the presence or absence of the complex in the sample based on the electrical signal. In some embodiments, the electrical signal further correlates with the presence or absence of the target molecule in the sample.

In some embodiments, the method further comprises a step of determining the presence or absence of the target molecule in the sample based on the electrical signal. In some embodiments, the method further comprises a step of determining the concentration of the complex or the target molecule in the sample.

The present invention also relates to a modified target molecule designed for nanopore detection. In some embodiments, the modified target molecule comprises a first attachment site for a driver molecule or a linker molecule, wherein the attachment site is generated by a first modification of a target molecule.

In some embodiments, the first modification comprises biotinylation, acetylation, methylation, summolation, glycosylation, phosphorylation, or oxidation. In some embodiments, the first modification comprises chemical or biological synthesis. In some embodiments, the first modification comprises a genetic engineering of the target.

In some embodiments, the first attachment site comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol. In some embodiments, the first attachment site can bind to the driver molecule or the linker molecule via a covalent bond, an intermediate linker, a Van der Waals bond, an electrostatic bond, a hydrophobic interaction, a pi-stacking interaction, an ionic bond, or another non-covalent electrostatic interaction.

In some embodiments, the target molecule comprises a biological therapeutic. In some embodiments, the biological therapeutic is a monoclonal antibody, a protein, a protease, a peptide, a sugar, a nucleic acid, or any combination thereof.

In some embodiments, the target molecule comprises a small molecule. In some embodiments, the small molecule is a small molecule therapeutic or small molecule diagnostic drug. In some embodiments, the small molecule is a small molecule therapeutic drug selected from the group consisting of an enzyme inhibitor or activator, a receptor antagonist or agonist, and a small molecule modifier of a cellular process or pathway. In some embodiments, the small molecule therapeutic drug is Lisinopril.

In some embodiments, the target molecule comprises an ingredient of a chemical or biological product, selected from the group consisting of a fertilizer, a construction or building material, and a dietary composition. In some embodiments, the ingredient is a nitrogenase. In some embodiments, the ingredient is boronic acid.

In some embodiments, the target molecule is an indicator selected from the group consisting of pH indicator, a toxin indicator, a chemical indicator, a pollutant indicator, or a temperature indicator.

In some embodiments, the modified target molecule maintains at least one function of the target molecule. In some embodiments, the at least one function is therapeutic, diagnostic, biological, chemical, or nutritional activity.

In some embodiments, the driver molecule is a single stranded deoxyribonucleic acid (ssDNA), a double stranded deoxyribonucleic acid (dsDNA), or a ribonucleic acid (RNA). In some embodiments, the driver molecule comprises more than 20 nucleotides.

In some embodiments, the modified target molecule further comprises a second attachment site for binding to a payload molecule. In some embodiments, the second attachment site is generated by a second modification of the target molecule or the modified target molecule. In some embodiments, the second modification comprises biotinylation, acetylation, methylation, summolation, glycosylation, phosphorylation, or oxidation. In some embodiments, the second modification comprises chemical or biological synthesis. In some embodiments, the second modification comprises a genetic engineering of the target.

In some embodiments, the second attachment site comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol. In some embodiments, the second attachment site can bind to the payload molecule via a covalent bond, an intermediate linker, a Van der Waals bond, an electrostatic bond, a hydrophobic interaction, a pi-stacking interaction, an ionic bond, or another non-covalent electrostatic interaction.

The present invention further relates to a method of detecting the modified target molecule. In some embodiments, the method comprises the steps of (a) obtaining a sample suspected to comprise the modified target molecule; (b) adding the driver molecule to the sample to generate a reaction product; (c) applying the reaction product to a nanopore device comprising at least one nanpore; (d) applying a voltage across said at least one nanopore; and (e) measuring an electrical signal through the pore that correlates with the presence or absence of the modified target molecule in the sample.

In some embodiments, the sample comprises a pharmaceutical composition. In some embodiments, the sample comprises a blood, urine, saliva, or tissue sample from a patient. In some embodiments, the sample comprises water, soil, air, sludge, petroleum, or a chemical or biological product.

In some embodiments, the modified target molecule is bound to one or more payload molecules, and wherein the electrical signal further correlates with the binding of said one or more payload molecules.

In some embodiments, the method further comprises the step of determining the presence or absence of the modified target molecule in the sample based on the electrical signal. In some embodiments, the method further comprises the step of determining the concentration of the modified target molecule in the sample.

Also provided herein are methods of analyzing data to detect the presence or absence of a target molecule in a, comprising obtaining an electrical signal from a nanopore device comprising a sample suspected of containing the modified target molecule described herein; and analyzing the electrical signal to detect the presence or absence of a signature correlated to the translocation of the target molecule through the nanopore, wherein the presence of the signature indicates the presence of the target molecule in said sample, and wherein the absence of the signature indicates the absence of the target molecule from said sample.

### 4. BRIEF DESCRIPTION OF THE DRAWINGS

Provided as embodiments of this disclosure are drawings that illustrate features by exemplification only, and not limitation.

FIG. 1 presents a target molecule (101) bound to a driver molecule (102) via a linker molecule (104) to facilitate translocation of the complex through a nanopore (106). The figure also presents back group molecules (105).

FIG. 2 illustrates a target molecule (101) that is not attached to a driver molecule (102). A nanpore device (106) senses an electrical signal that correlates with whether or not the driver molecule is bound to the target molecule.

FIGS. 3A-C illustrate electrical signals (time in the x-axis and current in the y-axis) unique to samples that translocate through the nanopore. The samples include a target molecule (101) bound to a driver molecule (102) through a linker molecule (104) (FIG. 3A); a driver molecule (102) alone (FIG. 3B); and a target molecule (101) alone (FIG. 3C).

FIG. 4 shows a chemical structure of a modified Lisonipril, wherein the Lisonipril molecule is covalently attached to a driver molecule (i.e., dsDNA) via an SMCC linker.

FIG. 5 provides data from mass spectrometry of Lisinopril labeled with an SMCC linker. The data verifies that Lisinopril is chemically attached to the linker molecule. The measured mass of 625.2 matches the predicted mass of 625-627.

FIG. 6 provides a graph plotting electrical signals from a target molecule (i.e., Lisinopril) attached to a driver molecule (i.e., 532 bp dsDNA).

FIG. 7 is a graph plotting electrical signals collected from a DNA-tetanus antibody complex (dots) or a DNA-PNA complex without an antibody (squares) that passes through a nanopore.

FIG. 8 presents a target molecule (101) bound to a driver molecule (102) through a linker molecule (104a). The target molecule is also bound to a payload molecule (103) via another linker molecule (104b).

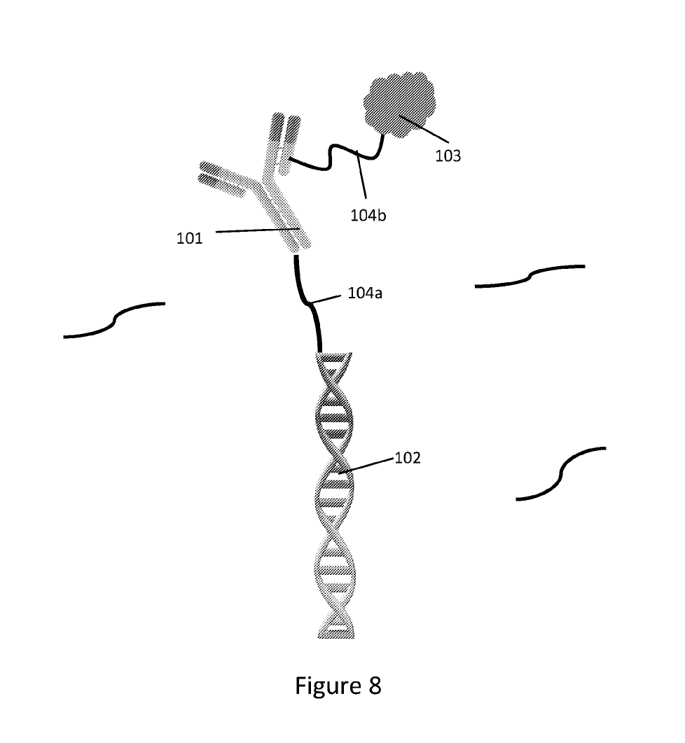

FIGS. 9A-B provide electrical signals unique to samples that translocate through the nanopore. The samples include a target molecule (101) bound to a driver molecule (102) and a payload molecule (103) via linker molecules (FIG. 9A) and a target molecule (101) bound to a driver molecule (102) via a linker molecule (FIG. 9B).

FIG. 10 provides a graph plotting electrical signals collected from a DNA-target-payload complex (dots) or a DNA-target complex without a payload (squares). The target is a peptide, and the payload is an antibody.

FIG. 11 provides a graph plotting electrical signals collected from a complex comprising a driver molecule (dsDNA) and a target molecule (biotin) without a payload (squares); and a complex comprising a driver molecule (dsDNA), a target (biotin), and a payload (an anti-biotin antibody) (reverse triangle).

FIG. 12 shows a target molecule (101) bound to a driver molecule (102) and a payload molecule (103a) via linkers (104a and b). The driver molecule is further bound to an additional payload molecule (103b, wavy box) for enhanced detection in a nanopore.

FIG. 13 illustrates translocation of the complex of FIG. 12 through a nanopore.

FIGS. 14A-C illustrate electrical signals unique to samples that translocate through a nanopore. The samples include a target molecule bound to a payload molecule and a driver molecule, wherein the driver molecule is bound to another payload molecule (FIG. 14A); a target molecule bound to a driver molecule and a payload molecule (FIG. 14B); and a target molecule bound to a driver molecule alone (FIG. 14C).

FIG. 15 shows a driver molecule (i.e., 5631 bp DNA) comprising two binding sites, one for a PNA-PEG and the other for a PNA-peptide.

FIGS. 16A-B provide a graph plotting electrical signals from different complexes passing through a nanopore. Dots represent signals from a driver molecule alone (DNA); squares and diamonds represent signals from a DNA and PNA-PEG (PP) complex; reverse triangles represent signals from a DNA and PNA-PEG (PP) with V3 loop (PV3B) complex; and stars represent signals from a DNA, PNA-PEG (PP) with V3 loop (PV3B) and HIV antibody complex.

Some or all of the figures are schematic representations for exemplification; hence, they do not necessarily depict the actual relative sizes or locations of the elements shown. The figures are presented for the purpose of illustrating one or more embodiments with the explicit understanding that they will not be used to limit the scope or the meaning of the claims that follow below.

### 5. DETAILED DESCRIPTION

### a. Interpretation of Terms

Throughout this application, the text refers to various embodiments of the present devices, compositions, systems, and methods. The various embodiments described are meant to provide a variety of illustrative examples and should not be construed as descriptions of alternative species. Rather, it should be noted that the descriptions of various embodiments provided may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entireties.

As used in the specification and claims, the singular form “a”, “an” and “the” include plural references unless the context clearly dictates otherwise. For example, the term “an electrode” includes a plurality of electrodes, including mixtures thereof.

As used herein, the term “comprising” is intended to mean that the systems, devices, and methods include the recited components or steps, but not excluding others. “Consisting essentially of” when used to define systems, devices, and methods, shall mean excluding other components or steps of any essential significance to the combination. “Consisting of” shall mean excluding other components or steps. Embodiments defined by each of these transition terms are within the scope of this invention.

All numerical designations, e.g., distance, size, temperature, time, voltage and concentration, including ranges, are approximations which are varied (+) or (−) by increments of 0.1. It is to be understood, although not always explicitly stated that all numerical designations are preceded by the term “about”. It also is to be understood, although not always explicitly stated, that the components described herein are merely exemplary, and that equivalents of such are known in the art.

As used herein, the term “target molecule” refers to a molecule of interest that may be modified for detection by a nanopore. The target molecule a physical dimension that can pass through a nanopore upon binding to the driver molecule.

As used herein, the term “linker molecule” refers to a molecule that can link between a target molecule and a driver molecule; between a target molecule and a payload molecule; or between a driver molecule and a payload molecule. The linker molecule a physical dimension that can pass through a nanopore upon binding to a target molecule and/or a driver molecule.

As used herein, the term “driver molecule” refers to a molecule sufficiently charged so that it can be captured in a nanopore in a solution under an applied voltage. Upon binding to a target molecule, the driver molecule can drive the target molecule into a nanopore. The driver molecule can comprise a single stranded deoxyribonucleic acid (ssDNA), a double stranded deoxyribonucleic acid (dsDNA), or a ribonucleic acid (RNA).

As used herein, the term “payload molecule” refers to a molecule with physical dimensions that facilitate generation of a unique electrical signal when captured in a nanopore. A payload molecule has a binding site for a target molecule or a driver molecule. In some embodiments, the payload molecule can be charged to facilitate passage through a nanopore.

As used herein, “a device comprising a nanopore that separates an interior space” shall refer to a device having a pore that comprises an opening within a structure, the structure separating an interior space into more than one volume or chamber.

As used herein, the term “electrical signal” encompasses a series of data collected on current, impedance/resistance, or voltage over time depending on configuration of the electronic circuitry. Conventionally, current is measured in a “voltage clamp” configuration; voltage is measured in a “current clamp” configuration, and resistance measurements can be derived in either configuration using Ohm's law V=IR. Impedance can also be generated by measured from current or voltage data collected from the nanopore device. Types of electrical signals referenced herein include current signatures and current impedance signatures, although various other electrical signatures may be used to detect particles in a nanopore.

As used herein, the term “nanopore” refers to an opening (hole or channel) of sufficient size to allow the passage of particularly-sized molecules. Voltage is applied to drive charged molecules through the nanopore.

As used herein, the term “sensor” refers to a device that collects a signal from a nanopore device. In many embodiments, the sensor includes a pair of electrodes placed at two sides of a pore to measure an ionic current across the pore when a molecule or other entity, in particular a target molecule, moves through the pore. In addition to the electrodes, an additional sensor, e.g., an optical sensor, can be used to detect an optical signal in the nanopore device. Other sensors can be used to detect such properties as current blockade, electron tunneling current, charge-induced field effect, nanopore transit time, optical signal, light scattering, and plasmon resonance.

As used herein, the term “current measurement” refers to a series of measurements of current flow at an applied voltage through the nanopore over time. The current is expressed as an x,y value where x represents a point in time, and y represents the amount of current impeded in the channel. Current measurement is an electrical signal related to current impedance/resistance and voltage (other electrical signals) through Ohm's law.

As used herein, the term “open channel” refers to the baseline level of current through a nanopore channel within a noise range where the current does not deviate from a threshold of value defined by the analysis software.

As used herein, the term “event” refers to a set of current impedance measurements that begins when the y value of a current measurement deviates from the open channel value by a defined threshold, and ends when the y value returns to within a threshold of the open channel value.

As used herein, the term “current impedance signature” refers to a collection of current measurements where the first such measurement begins when the value of y exceeds a given threshold defined by the software, and ends when the value of y returns past that same threshold. This threshold can be used to identify multiple signatures within an event (i.e., since a target molecule may have one or more molecules attached to it, an event may contain one or more unique signatures).

As used herein, the term “signature curve” refers to the product of a mathematical formula applied to all the x,y points in a single signature. This formula can be as simple as a simple average of all the points (yielding a single line at y), or as a moving average of every N number of points (yielding a simple curve), or another mathematical formula. Step fitting algorithms are another example of a formula to apply to each signature. The number of steps or their properties can be used to infer properties about the signature curve or curves. (See, e.g., C Raillon, P Granjon, M Graf, L J Steinbock, and A Radenovic. Fast and automatic processing of multi-level events in nanopore translocation experiments. Nanoscale, 4(16):4916, 2012, incorporated by reference in its entirety). Since nanopores are inherently non-deterministic, electrical signals can vary considerably each time the same type of molecule passes through. Therefore, the software that analyzes measurements can employ enough flexibility to assure a consistent signature curve each time the same molecule is read.

As used herein, the term “optical sensor” refers to an apparatus that captures light within a fixed field of view that may reside at or adjacent to the nanopore.

As used herein, the term “optical event” refers to a set of optical measurements captured by the sensor from a single target molecule. The optical measurements may be correlated with current impedance measurements to determine when a target molecule enters the nanopore.

As used herein, the term “optical measurement” refers to a value obtained by that optical sensor within a fixed period of time. This measurement may include, but not be limited to, one or more of individual values, such as color, luminescence, and intensity.

As used herein, the term “symbol” refers to the assembly of one or more optical signatures within an event so as to comprise a single abstraction. E.g., “red, green, red, green” may equate to the letter “A.”

### b. Target Molecules

The present invention provides devices and methods for adding a payload molecule and/or a driver molecule to a target molecule, such as a pharmaceutical compound. In certain embodiments, the target molecule comprises attachment sites to which a driver molecule can be attached. In certain embodiments, the target molecule comprises attachment sites to which a payload molecule can be attached. In some embodiments, the attachment sites are part of the chemical makeup of the target molecule, including but not limited to, reactive groups on an amino acid, a double stranded deoxyribose nucleic acid (dsDNA), a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

In some embodiments, the target molecule is a biological therapeutic, including a monoclonal antibody, a protein, a protease, a peptide, a sugar, a nucleic acid, or any combination thereof.

In some embodiments, the target molecule comprises a small molecule. In some embodiments, the small molecule is a small molecule therapeutic, including but not limited to, enzyme inhibitors or activators, receptor antagonists or agonists, or other small molecule modifiers of cellular processes or pathways. In some embodiments, the small molecule is Lisinopril. In some embodiments, the small molecule is a small molecule diagnostic drug.

In some embodiments, the target molecule is a synthetic molecule. In some embodiments, the target molecule comprises an ingredient of a chemical or biological product, selected from the group consisting of fertilizer, construction or building materials, and a dietary composition. In some embodiments, the ingredient is an essential component of the chemical or biological product having a specific function. In some embodiments, the ingredient is an enzyme. In some embodiments, the enzyme is nitrogenase. In some embodiments, the ingredient is boronic acid.

In some embodiments, the target molecule is an indicator that changes its conformation, structure, or stability in response to a physical condition. In some embodiments, the indicator is a pH indicator, a toxin indicator, a chemical indicator, a pollutant indicator, or a temperature indicator.

### c. Linker Molecules

In some embodiments, the target molecule and driver molecules are covalently coupled through the attachment site via a linker molecule.

In some embodiments, the linker molecule comprises a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid (PNA), a polyethylene glycol (PEG), a dendrimer, a synthetic polymer, or any combination thereof.

In some embodiments, the linker molecule is a peptide, a valine-citrulline linker, a maleimidocaproyl (mc) linker, an [N-maleimidomethyl]cyclohexane-1-carboxylate (MCC) linker, a succinimidyl 4[N-maleimidomethyl]cyclohexane-1-carboxylate (SMCC) linker, a PEG-based linker, a hydrazone linker, an N-succinimidyl-4-(2-pyridyldithio) butanoate (SPDB) linker, or a carbonate linker, or any other homobifunctional or heterobifunctional molecule.

In some embodiments, the linker molecule is a charged molecule that can facilitate capture of the target molecule in a nanopore.

### d. Payload Molecules

In some embodiments, the target molecule is modified by one or more payload molecules that provide a unique electrical signal as it translocates into or through the nanopore, so as to enable discrimination from other target/payload molecules or background molecules present in the sample. The payload molecule is configured to allow detection of the target molecule in a nanopore. These payload molecules can be identified and distinguished from each other by their current impedance when passing through the nanopore. This current impedance is affected by width, length, size and/or charge of the payload molecule. The payload molecules can differ from each other by size, shape, or charge. Thus, each payload molecule may provide a unique electrical signal upon passage through the nanopore, allowing identification of a target molecule bound to the payload molecule in a nanopore.

Since the payload molecules can be modified by parameters such as width, length, size, and/or charge, the compositions and methods described herein can be performed with pores of varying size, including larger pores, which are easier and cheaper to manufacture than smaller nanopore devices. A greater size of a detectable marker results in a greater change in current flow through the pore, or current impedance, compared to an unlabeled compound.

In some embodiments, the payload molecule is a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid, a polyethylene glycol (PEG), a dendrimer, or any combination thereof. In some embodiments, the payload molecule is linked to the target molecule via covalent bond, intermediate linker, or non-covalent electrostatic interaction.

In some embodiments, a target molecule, a driver molecule or a linker molecule has a binding site to a payload molecule. In some embodiments, a target molecule, a driver molecule or a linker molecule contains additional attachment site(s) capable of linking additional signal differentiating payload molecules, allowing further discrimination or enhanced identification of a target molecule in a sample. In some embodiments, one or more of the target, driver, and payload molecules are further modified with additional payload molecules. In some embodiments, the additional payload molecule is linked directly to the attachment site by covalent bond, intermediate linker, Van der Waals bond, electrostatic bond, hydrophobic bond, pi-stacking, ionic bond, or other non-covalent electrostatic interaction.

### e. Driver Molecules

The driver molecule is configured to facilitate capture of the target molecule in a nanopore. In some embodiments, the driver molecule can be captured by voltage into the nanopore. In some embodiments, the target molecule and driver molecule are directly linked together through the attachment site by a covalent bond. In some embodiments, the target and driver molecules are not linked by covalent bond, but instead by a non-covalent linkage, including an electrostatic interaction, a hydrogen bond, a salt bridge, a van der Waals interaction, a cation-pi interaction or a hydrophobic interaction. In some embodiments, the driver molecule comprises a single stranded deoxyribonucleic acid (ssDNA), a double stranded deoxyribonucleic acid (dsDNA), a ribonucleic acid (RNA), a peptide, a protein, a carbohydrate, or a combination thereof. In some embodiments, the driver molecule is one or more synthetic molecules, e.g., dendrimer, a nano/micro particle, a polyethylene glycol (PEG), a peptide nucleic acid (PNA) or other synthesized polymer. In some embodiments, the target molecule is in the same formulation but not associated with the attachment site of the driver molecule. In some embodiments, the driver molecule provides a current impedance as it translocates into or through the nanopore, thereby allowing one to infer the presence or absence of the target molecule.

The driver molecule is configured to provide an appropriate diffusion coefficient and electrical mobility upon binding to a target molecule. In some embodiments, electrical mobility of a driver molecule is measured by techniques known in the art. In some embodiments, the electrical mobility is determined by gel electrophoresis, wherein native charge is measured under particular static conditions, which can include a particular ionic strength buffer, pH and driving force (e.g. TEA buffer pH 7.5 and 50 V driving force). In some embodiments, the electrical mobility is determined by isoelectric focusing (IEF), which measures analyte mobility based on its charge in a variable “zoned” buffer system. Under this method, different charged variants stop migrating when they enter a region of pH that matches the analytes isoelectric point. By analyzing differently migrated “bands on a gel” one can infer the different charged states of the molecule. In some embodiments, Ion Exchange Chromatography (IEX) measures mobility off a solid support based on analyte charge. Here, a target molecule(s) is bound to a column and eluted off using a pH gradient. When the pH matches the isoelectric point of the molecule, the analyte no longer binds to the column, and is eluted off and subsequently detected. In some embodiments, the electrical mobility is determined based on MALDI-TOF (time of flight) determined by a mass to charge ratio. The greater the charge and less mass, the quicker it travels to a detector. Conversely, less charge and greater mass results in a greater “flight time”. Molecules are identified using their unique “fly time” and comparing to standards.

The driver molecule is configured to have a physical dimension sufficiently small to pass through a nanopore. In some embodiments, the driver molecule comprises a polynucleotide with at least 20 bps. In some embodiment, the driver molecule is configured to have a physical dimension sufficiently small to pass through a nanopore when it binds to a target molecule, a payload molecule or a plurality of payload molecules.

### f. Target Molecule Modifications

The present disclosure provides methods and systems for target molecule modification and detection. In certain embodiments, provided herein are methods for modifying a target molecule for the purpose of detecting and/or quantifying the presence or absence of the target molecule in a sample. In certain embodiments, modified target molecules may be detected in a nanopore device.

In some embodiments, the target molecule is modified to comprise an attachment site for a target molecule. In some embodiments, the target molecule is further modified to comprise an attachment site for a payload molecule. In some embodiments, the attachment site is generated by biological or chemical modification of the target molecule. In some embodiments, the chemical modification is biotinylation, acetylation, methylation, summolation, glycosylation, phosphorylation, or oxidation. In some embodiments, the attachment site is introduced by molecular, biological or biochemical methods. In some embodiments, the attachment site is introduced by enzymatic reaction. In some embodiments, the attachment site is introduced by genetic engineering.

In some embodiments, the attachment site comprises a reactive group on an amino acid, a deoxyribose nucleic acid (DNA), a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol. In some embodiments, the attachment site can bind to a driver molecule or a payload molecule via a covalent bond, an intermediate linker, Van der Waals bond, electrostatic bond, hydrophobic bond, pi-stacking, ionic bond, or other non-covalent electrostatic interaction.

In some embodiments, a target molecule comprising an attachment site is biologically or chemically synthesized. In some embodiments, the synthesis involves biotinylation, acetylation, methylation, summolation, glycosylation, phosphorylation, oxidation or other enzymatic reaction. In some embodiments, the attachment site comprises a reactive group on an amino acid, a deoxyribose nucleic acid (DNA), a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol. In some embodiments, the attachment site can bind to a driver molecule or a payload molecule via a covalent bond, an intermediate linker, Van der Waals bond, electrostatic bond, hydrophobic bond, pi-stacking, ionic bond, or other non-covalent electrostatic interaction.

In some embodiments, the modified target molecule maintains at least one function of the target molecule. In some embodiments, the modification of the target molecule does not change an activity of the target molecule as a biologic therapeutic, a small molecule drug, and an ingredient of a chemical or biological product.

### g. Target-Driver Complexes

In some embodiments, a target molecule and a driver molecule are covalently coupled. In some embodiments, a complex comprising a target molecule and a driver molecule is generated by a chemical reaction making a covalent bond between the target molecule and the driver molecule. In some embodiments, a complex comprising a target molecule and driver molecule is synthesized by a chemical, biological, molecular and genetic reaction.

In some embodiments, the complex comprising a target molecule and a driver molecule is used as a pharmaceutical composition for treatment or diagnotic purposes. In some embodiments, the complex comprising a target molecule and a driver molecule is used as an indicator, such as a pH indicator, a toxin indicator, a chemical indicator, a pollutant indicator, or a temperature indicator. In some embodiments, the complex comprising a target molecule and a driver molecule is used as an ingredient of a chemical or biological product such as fertilizer, construction or building materials, and a dietary composition.

### h. Target-Linker Complexes

In some embodiments, the target molecule and driver molecules are covalently coupled to a linker molecule. In some embodiments, a complex comprising a target molecule and a linker molecule is generated by a chemical reaction making a covalent bond between the target molecule and the linker molecule. In some embodiments, a complex comprising a target molecule and linker molecule is synthesized by a chemical, biological, molecular and genetic reaction.

In some embodiments, the linker comprises a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid (PNA), a polyethylene glycol (PEG), a dendrimer, a synthetic polymer, or any combination thereof. In some embodiments, the linker comprises at least one of a peptide, a valine-citrulline linker, a maleimidocaproyl (mc) linker, an [N-maleimidomethyl]cyclohexane-1-carboxylate (MCC) linker, a succinimidyl 4[N-maleimidomethyl]cyclohexane-1-carboxylate (SMCC) linker, a PEG-based linker, a hydrazone linker, an N-succinimidyl-4-(2-pyridyldithio) butanoate (SPDB) linker, or a carbonate linker, or any other homobifunctional or heterobifunctional molecule.

In some embodiments, the complex comprising a target molecule and a linker molecule is used as a pharmaceutical composition for treatment or diagnotic purposes. In some embodiments, the complex comprising a target molecule and a driver molecule is used as an indicator, such as a pH indicator, a toxin indicator, a chemical indicator, a pollutant indicator, or a temperature indicator. In some embodiments, the complex comprising a target molecule and a driver molecule is used as an ingredient of a chemical or biological product such as fertilizer, construction and building materials, and a dietary composition.

### i. Methods of Detection

In some embodiments, the present technology provides a method for identifying a target molecule in a sample. The method entails (a) loading a sample suspected to comprise a target molecule into a device with a pore that separates and connects two volumes, under conditions that allow a target molecule (e.g., a pharmaceutical drug bound to a detectable payload molecule) to translocate through the pore from one volume to the other volume, and (b) collecting an electrical signal correlated to the passage of the target molecule through the nanopore. Using the electrical signal, events correlated to detectable molecules translocating through the nanopore may be collected and analyzed to identify electrical signals correlated with a target molecule attached to the payload molecule.

In some embodiments, the method entails (a) obtaining a sample suspected to comprise a modified target molecule, (b) adding the driver molecule to the sample to generate a reaction product, (c) applying the reaction product to a nanopore device, (d) applying a voltage across each pore, and (e) measuring an electrical signal through the pore that correlates with the presence or absence of the modified target molecule in the sample.

In some embodiments, the method further entails determining the presence or absence of a target molecule or a complex comprising a target molecule in the sample based on an electrical signal. In some embodiments, the method further entails determining the concentration of a target molecule or a complex comprising a target molecule in the sample based on an electrical signal.

An “electrical signal” can include current measurement creating a current signature from the translocation through the pore of one, or alternatively two or more payload molecules attached to a target molecule at a time.

In some embodiments, a plurality of target molecules can be detected using a plurality of unique payload molecules, wherein each target molecule bound to a unique payload molecule generates a unique electrical signal used for identification of each distinct target molecule. For instance, payload molecule A may provide one unique electrical signal, whereas payload molecule B can form a different unique electrical signal.

In each of these above scenarios, the nanopore device can be suitably configured to identify each unique electrical signal generated each unique payload molecule bound to its respective target molecule.

In some embodiments, the sample is a pharmacological composition. In some embodiments, the sample is a mixed sample, e.g., a blood or urine sample obtained from a patient. In some embodiments, the sample is a blood, urine, saliva, or tissue sample from a patient. In some embodiments, the sample is obtained from a subject who has been administered with a target. In some embodiments, the sample is obtained from a human patient.

In some embodiments, the sample is water, soil, air, sludge, petroleum, or a chemical or biological product. Methods and compositions for analyte detection are disclosed in PCT Publication WO/2014/182634, incorporated by reference in its entirety.

In a nanopore experiment, a population of current impedance events is generated. Mathematical modeling is used to pick out our target molecules from background within a degree of confidence. However, a mixed sample, e.g., blood that has not been processed, has a large population of background molecules that produce electrical signals that overlap with those of a target. A high error rate may be introduced by these molecules, affecting the reliability of the nanopore to detect target molecules. One mechanism to improve reliability, as disclosed herein, is to attach a one or payload molecules to a target molecule to provide a unique electrical signal that can be used to identify the presence and/or identity of a target molecule bound to the payload molecule that has translocated through a nanopore. Therefore, provided are improved methods and compositions for detecting modified target molecules attached to one or more payload molecules in a bulk sample using a nanopore.

The formed complex including the target molecule and one or more driver molecules and/or payload molecules can be detected using a device that includes a nanopore (or simply, pore), and a sensor. The pore is a nano-scale or micro-scale opening in a structure separating two volumes. The sensor is configured to identify objects passing through the pore. For example, in some embodiments, the sensor identifies objects passing through the pore by detecting a change in a measurable parameter, wherein the change is indicative of an object passing through the pore. This device is referred throughout as a “nanopore device.” In some embodiments, the nanopore device includes electrodes connected to power sources, for moving the target molecule from one volume to another, across the pore. As the target molecule can be charged or bound to a driver molecule comprising a charge. By generating a potential or voltage across the pore the movement of the target molecule or target molecule bound to a driver molecule is facilitated and controlled. In certain embodiments, the sensor comprises a pair of electrodes, which are configured both as a sensor to detect the passage of objects through the nanopore by reading current, and to provide a voltage, across the pore. In certain embodiments, a voltage-clamp or a patch-clamp is used to simultaneously supply a voltage across the pore and measure the current through the pore.

Detection of the target molecule bound to the payload molecule can be carried out by various methods. In one aspect, translocation of the target molecule through the pore under an applied voltage in a solution comprising a predetermined salt (e.g., KCl) concentration will generate a unique detectable electrical signal. The electrical signals can be differentiated from one another by the amount of the current shift (height) and/or the duration of the current shift (width), or by any other feature in the signal that differentiates the electrical signature.

In some embodiments, the sensor comprises electrodes, which are connected to power sources and can detect the current. Either one or both of the electrodes, therefore, serve as a “sensor.” In this embodiment, a voltage-clamp or a patch-clamp is used to simultaneously supply a voltage across the pore and measure the current through the pore.

In some aspects, a probe is added to the target molecule to aid detection. This probe is capable of binding to the payload molecule or driver molecule attached to the target molecule. In one aspect, the probe has a charge, either negative or positive, to facilitate detection in a nanopore. In another aspect, the probe adds size to facilitate detection in a nanopore. In another aspect, the probe includes a detectable label, such as a fluorophore.

### j. Optical Detection

The payload molecules may be detected by methods known in the art as an alternative to the use of current impedance. In some embodiments, payload molecules may include, e.g., fluorescent dyes (e.g., Cy5®, Cy3®, FITC, rhodamine, lanthamide phosphors, Texas red), 32P, 35S, 3H, 14C, 125I, 131I, electron-dense reagents (e.g., gold), enzymes as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other payload molecules include labels or oligonucleotides capable of forming a complex with the corresponding receptor or oligonucleotide complement, respectively.

In some aspects, the payload molecule is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

Suitable fluorescent moieties include the following fluorophores known in the art: 4-acetamido-4′-isothiocyanatostilbene-2,2′disulfonic acid acridine and derivatives: acridine, acridine isothiocyanate, Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS), 4-amino-N-(3-vinyl sulfonyl) phenyl]naphthalimide-3,5 disulfonate (Lucifer Yellow VS), N-(4-anilino-1-naphthyl) maleimide, anthranilamide, Black Hole Quencher™ (BHQ™) dyes (biosearch Technologies), BODIPY® R-6G, BOPIPY® 530/550, BODIPY® FL Brilliant Yellow; coumarin and derivatives: coumarin, 7-amino-4-methylcoumarin (AMC, Coumarin 120); 7-amino-4-trifluoromethylcouluarin (Coumarin 151), Cy2®, Cy3®, Cy3.5®, Cy5®, Cy5.5®; Cyanosine 4′,6-diaminidino-2-phenylindole (DAPI) 5′, 5″-dibromopyrogallol sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid 5 [dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC), Eclipse™ (Epoch Biosciences Inc.); eosin and derivatives: eosin, eosin isothiocyanate; erythrosin and derivatives: erythrosin B, erythrosin isothiocyanate, ethidium fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide; pyrene and derivatives: pyrene, pyrene butyrate, succinimidyl 1-pyrene butyrate, QSY® 7, QSY® 9, QSY® 21, QSY® 35 (Molecular Probes), Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

Other fluorescent nucleotide analogs can be used, see, e.g., Jameson et al., 278 Meth. Enzymol. 363-390 (1997); Zhu et al., 22 Nucl. Acids Res. 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

### k. Nanopore Devices

A nanopore device, as provided, includes at least a pore that forms an opening in a structure separating an interior space of the device into two volumes, and at least a sensor configured to identify objects (for example, by detecting changes in parameters indicative of objects) passing through the pore. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some aspects, each pore is at least about 100 nm, 200 nm, 500 nm, 1000 nm, 2000 nm, 3000 nm, 5000 nm, 10000 nm, 20000 nm, or 30000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore(s) in the nanopore device are of a larger scale for detecting large microorganisms or cells. In one aspect, each pore has a size that allows a large cell or microorganism to pass. In one aspect, each pore is at least about 100 nm in diameter. Alternatively, each pore is at least about 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, or 5000 nm in diameter.

In one aspect, the pore is no more than about 100,000 nm in diameter. Alternatively, the pore is no more than about 90,000 nm, 80,000 nm, 70,000 nm, 60,000 nm, 50,000 nm, 40,000 nm, 30,000 nm, 20,000 nm, 10,000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 100 nm and about 10000 nm, or alternatively between about 200 nm and about 9000 nm, or between about 300 nm and about 8000 nm, or between about 400 nm and about 7000 nm, or between about 500 nm and about 6000 nm, or between about 1000 nm and about 5000 nm, or between about 1500 nm and about 3000 nm.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, each pore has a depth (i.e., a length of the pore extending between two adjacent volumes) that is least about 0.3 nm. Alternatively, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm.

In one aspect, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm.

In one aspect, the pore has a depth that is between about 1 nm and about 100 nm, or alternatively, between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore device further includes means to move a target molecule across the pore and/or means to identify objects that pass through the pore. Further details are provided below, described in the context of a two-pore device.

Compared to a single-pore nanopore device, a two-pore device can be more easily configured to provide good control of speed and direction of the movement of the target molecule across the pores.

In certain embodiments, the nanopore device includes a plurality of chambers, each chamber in communication with an adjacent chamber through at least one pore. Among these pores, two pores, namely a first pore and a second pore, are placed so as to allow at least a portion of a target molecule to move out of the first pore and into the second pore. Further, the device includes a sensor capable of identifying the target molecule during the movement. In one aspect, the identification entails identifying individual components of the target molecule. In another aspect, the identification entails identifying fusion molecules and/or target analytes bound to the target molecule. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple target molecules to move from one chamber to the next simultaneously. Such a multi-pore design can enhance throughput of target molecule analysis in the device.

For instance, in a three-chamber two-pore device (a “two-pore” device), each of the chambers can contain an electrode for connecting to a power supply so that a separate voltage can be applied across each of the pores between the chambers.

In accordance with an embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

In one aspect, in devices comprising two or more pores, the pores are spaced apart at a distance that is between about 10 nm and about 1000 nm. In some aspects, the distance between the pores is greater than 1000 nm, 2000 nm, 3000 nm, 4000 nm, 5000 nm, 6000 nm, 7000 nm, 8000 nm, or 9000 nm. In some aspects, the pores are spaced no more than 30000 nm, 20000 nm, or 10000 nm apart. In one aspect, the distance is at least about 10 nm, or alternatively, at least about 20 nm, 30 nm, 40 nm, 50 nm, 60 nm, 70 nm, 80 nm, 90 nm, 100 nm, 150 nm, 200 nm, 250 nm, or 300 nm. In another aspect, the distance is no more than about 1000 nm, 900 nm, 800 nm, 700 nm, 600 nm, 500 nm, 400 nm, 300 nm, 250 nm, 200 nm, 150 nm, or 100 nm.

In yet another aspect, the distance between the pores is between about 20 nm and about 800 nm, between about 30 nm and about 700 nm, between about 40 nm and about 500 nm, or between about 50 nm and about 300 nm.

The two pores can be arranged in any position so long as they allow fluid communication between the chambers and have the prescribed size and distance between them. In one aspect, the pores are placed so that there is no direct blockage between them. Still, in one aspect, the pores are substantially coaxial.

In one aspect, the nanopore device is connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies.

In some aspects, the power supply or supplies are configured to apply a first voltage V₁ across a first pore, and a second voltage across a second pore. In some embodiments, the two pores are in series. In some embodiments, the first pore is between an upper chamber and a middle chamber, and the second pore is between a middle chamber and a lower chamber. In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

Additional embodiments of the nanopore device and mechanisms of use for detection are described in PCT Publication WO2013/012881 and PCT Publication WO2013/074546, each incorporated by reference in its entirety.

### l. Sensors

As discussed above, in various aspects, the nanopore device further includes one or more sensors to carry out the identification of the binding status of the binding motifs.

The sensors used in the device can be any sensor suitable for identifying a target molecule. For instance, a sensor can be configured to identify the target molecule by measuring a current, a voltage, a pH value, an optical feature, or residence time associated with the target molecule or bound payload molecule. In other aspects, the sensor may be configured to identify one or more individual components of the target molecule or one or more components bound to the target molecule (i.e., the payload molecule). The sensor may be formed of any component configured to detect a change in a measurable parameter where the change is indicative of the target molecule or payload molecule. In one aspect, the sensor includes a pair of electrodes placed at two sides of a pore to measure an ionic current across the pore when a molecule or other entity, in particular a target molecule, moves through the pore. In certain aspects, the ionic current across the pore changes measurably when a target molecule segment passing through the pore is bound to a payload molecule. Such changes in current may vary in predictable, measurable ways corresponding with, for example, the presence, absence, and/or size of the one or more payload molecules present and bound to the target molecule.

In a preferred embodiment, the sensor comprises electrodes which apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. The result when a molecule translocates through a nanopore in an electrical field (e.g., under an applied voltage) is an electrical signal that may be correlated to the molecule passing through the nanopore upon further analysis of the current signal.

When residence time measurements from the electrical signal are used, the size of the component can be correlated to the specific component based on the length of time it takes to pass through the sensing device.

In an embodiment, a sensor is provided in the nanopore device that measures an optical feature of the target molecule, a component (or unit) of the target molecule, or a component bound to the target molecule (e.g., a payload molecule or a driver molecule). One example of such measurement includes the identification of an absorption band unique to a particular unit by infrared (or ultraviolet) spectroscopy.

In some embodiments, the sensor is an electric sensor. In some embodiments, the sensor detects a fluorescent detection means when the target analyte or the detectable label passing through has a unique fluorescent signature. A radiation source at the outlet of the pore can be used to detect that signature.

m. Analysis of Data from Nanopore Detection:

In some embodiments, the method of identifying a target molecule is performed on a device that contains one or more nanopores. A microfluidic channel may be included in the device that allows sample to enter into the reservoir chamber. A voltage is then applied to the sample mixture, causing charged molecules, such as target molecules bound to a driver molecule, to pass through the nanopore. As molecules pass through the pore, events are generated, and the data is analyzed by the software to discern the presence of known signature curves correlating to the presence of a target molecule.

In some embodiments, the electrical signals are generated upon passage of a complex comprising a target molecule through a nanopore. In some embodiments, the electrical signals are correlated with presence or absence of the target molecule within the sample. In some embodiments, the electrical signals are correlated with the concentration of the target molecule within the sample.

In another embodiment optical signals may be used instead of current impedance measurements to discern the presence of a target molecule. Voltage is applied to drive charged molecules through the nanopore. An optical sensor is used in the device to capture an optical measurement within a fixed field of view that may reside at or adjacent to the nanopore. The optical measurement comprises a measure of light detected within a fixed period of time. This measurement may include, but not be limited to, one or more of individual values, such as color, luminescence, and intensity. The method can be used to detect a target molecule that has been modified in such a manner to generate an optical signal that an optical sensor will detect, providing a particular optical measurement.

An “optical event” is a set of optical measurements captured by the sensor from a single target molecule translocation that may contain one or more payload molecules. An optical signature is a collection of optical measurements within an optical event where the software analyzes them in such a manner that it determines it has read a unique abstract value.

In several of the embodiments, the electrical or optical signal provided may be compared against a database that correlates a target molecule with an electrical or optical signal. This target molecule may be any of the entities discussed herein as capable of being detected via current impedance upon translocation through the nanopore, or other methods of detection, such as optical measurements. A database may be generated by reading the electrical signals provided by a homogenous population. Analysis of a homogenous population of target molecules bound to a payload molecule, which may further be bound to a driver molecule, is useful for assessing the variation in signal pattern generated and determining a reference signal for that coded molecule.

### n. Applications

In some embodiments, the present invention is applied to detect a presence or an absence of a target molecule in a sample. In some embodiments, the present invention is applied to measure a concentration of a target molecule in a sample. In some embodiments, the present invention is applied to detect a presence or an absence of a complex comprising a target molecule. In some embodiments, the present invention is applied to measure a concentration of a complex comprising a target molecule in a sample. In some embodiments, the present invention is applied to detect a physical property of a target molecule or a complex comprising a target molecule.

In some embodiments, the sample is a pharmacological composition. In some embodiments, the sample is a blood, urine, saliva, or tissue sample from a patient. In some embodiments, the sample is a mixed sample, e.g., a blood or urine sample obtained from a patient. In some embodiments, the sample is obtained from a subject who has been administered with a target. In some embodiments, the sample is obtained from a human patient.

In some embodiments, the sample is water, soil, air, sludge, petroleum, or a chemical or biological product.

In some embodiments, nanopore detection of a target molecule or a complex comprising a target molecule provides information about a composition of a sample. In some embodiments, nanopore detection of a target molecule or a complex comprising a target molecule provides information about a physical condition of the sample, such as pH, a composition, or temperature. In some embodiments, nanopore detection of a target molecule or a complex comprising a target molecule provides information about stability of the target molecule. In some embodiments, nanopore detection of a target molecule or a complex comprising a target molecule provides information about the origin of the sample.

### 6. EXAMPLES

### a. Example 1

### i. Target-Linker-Driver Complex

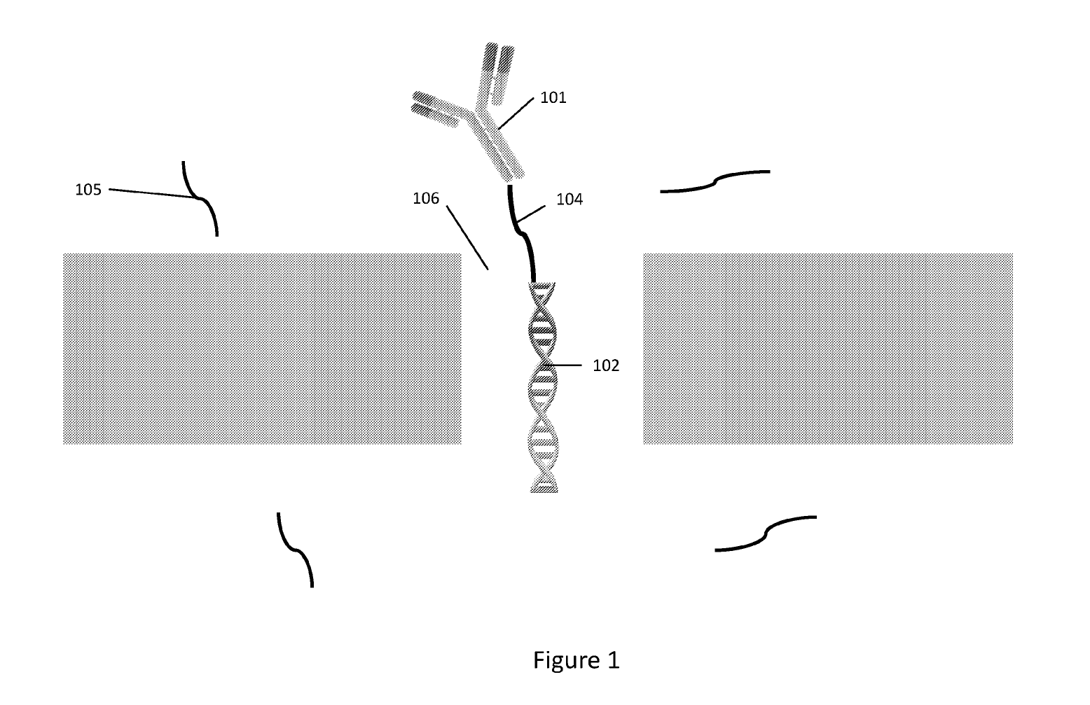

FIG. 1 illustrates an embodiment of the disclosed methods and systems. More specifically, the figure discloses a target molecule 101 bound to a driver molecule 102 via a linker molecule 104. The driver molecule 102 is charged to facilitate translocation of the target molecule 101 through the nanopore 106 under an applied voltage. Also shown in FIG. 1 are background molecules 105 that may or may not generate a detectable signal upon capture in the nanopore.

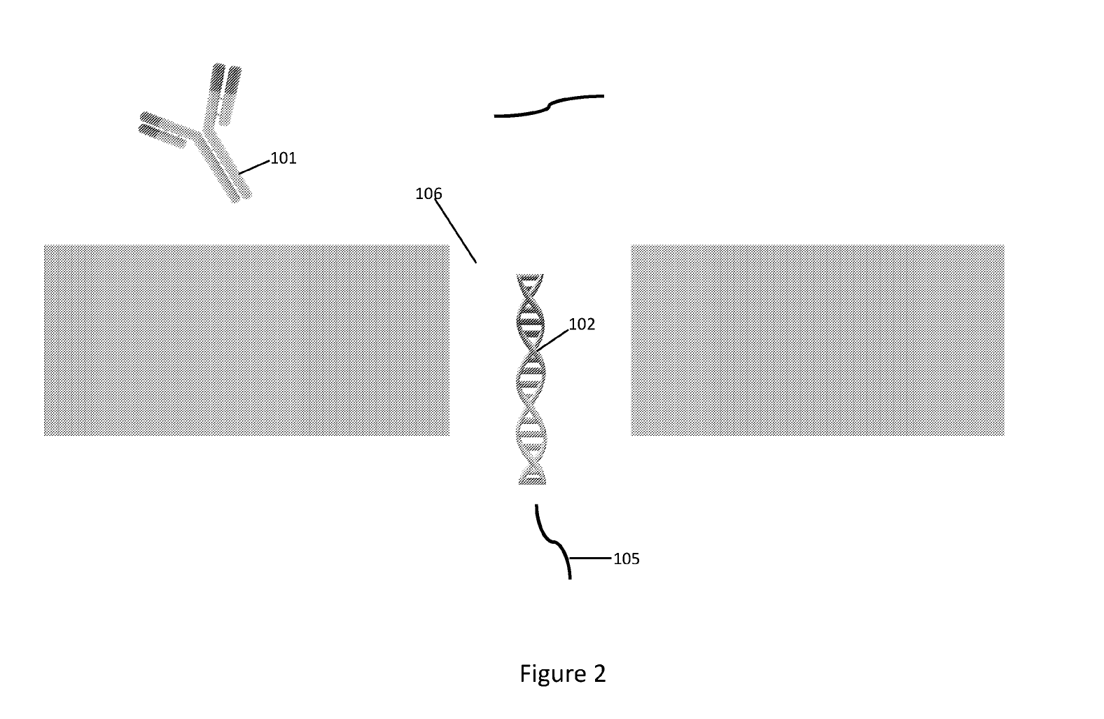

Under certain conditions, the driver molecule 102 can be separated from the target molecule 101 as provided in FIG. 2. If this happens, the driver molecule 102 passes through the nanopore without the target molecule.

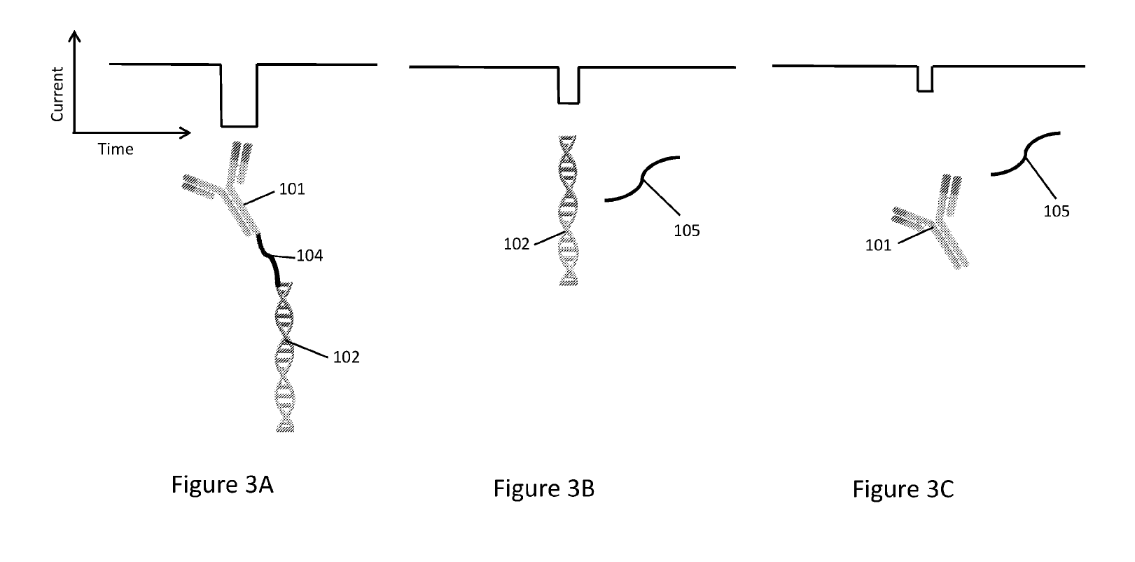

As illustrated in FIG. 3, electrical signals generated upon translocation of the driver molecule alone (FIG. 3B) or translocation of the target molecule alone (FIG. 3C) are different from electrical signals generated upon translocation of the driver-target complex (FIG. 3A). In this case, the target molecule bound to the driver molecule provides an electrical signal that is longer in duration and/or has a greater change from an open channel signal (FIGS. 3A-C).

### ii. Nanopore Detection of Target (Lisinopril)-Linker (SMCC)-Driver (DNA) Complex

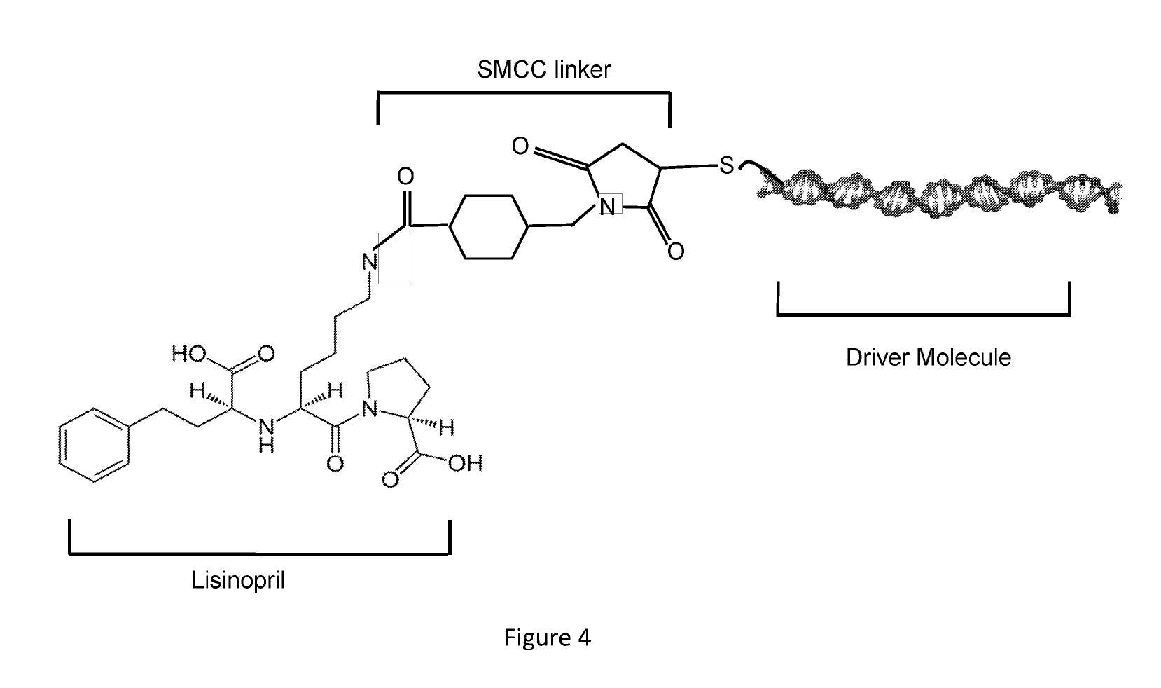

The present invention provides a drug compound attached to a driver molecule such that it is detectable in a nanopore based on current impedance. Lisinopril, an angiotensin conversion enzyme inhibitor, is a small molecule drug sold by Astrazeneca for treatment of hypertension. Given its small size, 405 Da, Lisinopril is too small to be detected in a nanopore. However, when conjugated to a driver molecule, Lisinopril can be shuttled through the nanopore allowing detection. FIG. 4 shows a chemical structure of Lisinopril modified to covalently bind to an SMCC linker, which binds to a dsDNA driver molecule.

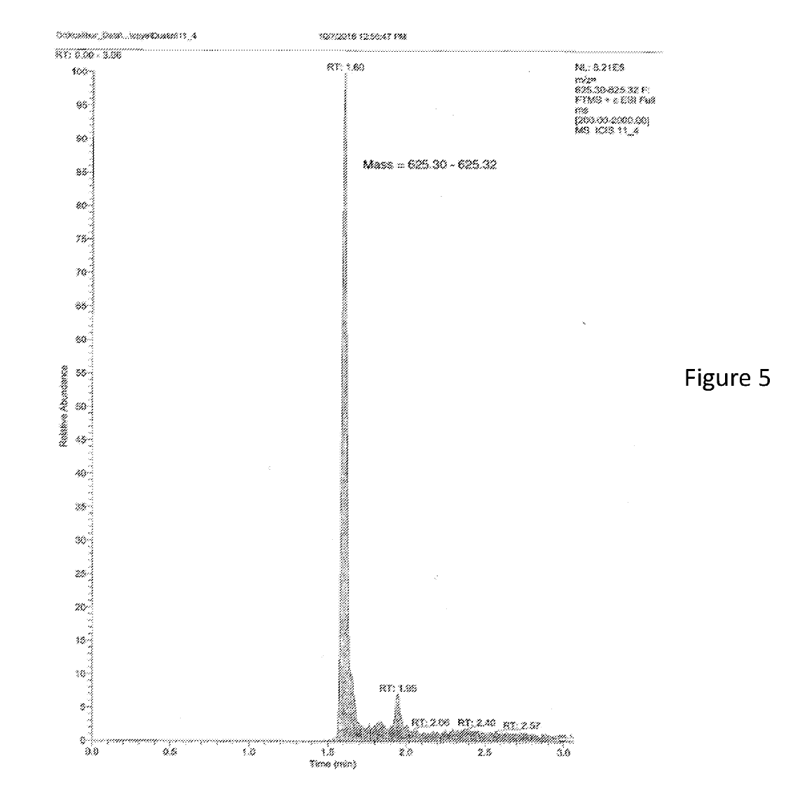

Specifically, we modified Lisinopril to comprise a free amine for attachment to NETS-esters containing molecules. We linked the modified Lisinopril to thiolated DNA via an SMCC linker, the NETS-ester reactive group of the SMCC linker reacts with the free amine in Lisinopril, while the maleimide group on the linker reacts with the 5′ thiol on the DNA. We confirmed SMCC labeling of the Lisinopril via ESI-mass spec, the measured mass of 625.2 (FIG. 5) matched the predicted mass of 625-627. This Lisinopril-SMCC adduct was then reacted with the thiolated DNA resulting in the Lisinopril-DNA (or Target-Driver) complex.

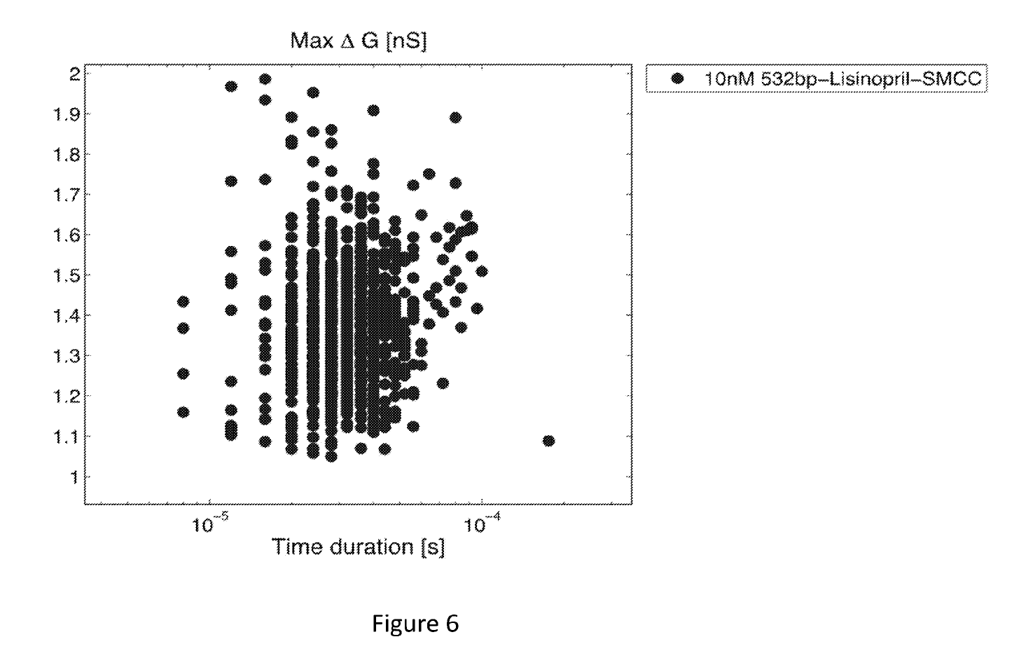

The DNA driver molecule facilitated translation and detection of the Lisinopril in a nanopore device. The DNA-Lisinopril complex generated easily detectable electrical signals as plotted in FIG. 6 (dots). Lisinopril without the driver gave no signal when passed through the pore. DNA-Lisinopril at a 10 nM concentration provides 144 events per minute that range from 1-2 nS in maximum conductance shift and 10-5 to 10-4 s in duration. Without attachment to the 532 bp dsDNA driver molecule, Lisinopril is not detectable with a nanopore.

### iii. Nanopore Detection of Target (Monoclonal Antibody)-Linker (PNA-SMCC)-Driver (DNA) Complex

The present invention also provides a method for detecting a target drug biologic by covalently coupling the target to a domain that binds a driver molecule to usher it through a pore under an electrophoretic current. Specifically, a monoclonal antibody drug that can bind and neutralize a pathogenic protein in Tetanus bacteria (Tetanus toxin) is covalently conjugated to a PNA, through an SMCC linker, that binds a DNA driver molecule in a sequence specific location. The complex (Driver-Drug) is clearly detectable in the pore, whereas the Drug by itself and the Driver by itself are not detectable. Thus, only the DRIVER-DRUG complex can be detected in the pore reducing to practice the concept in FIG. 1 of this patent.

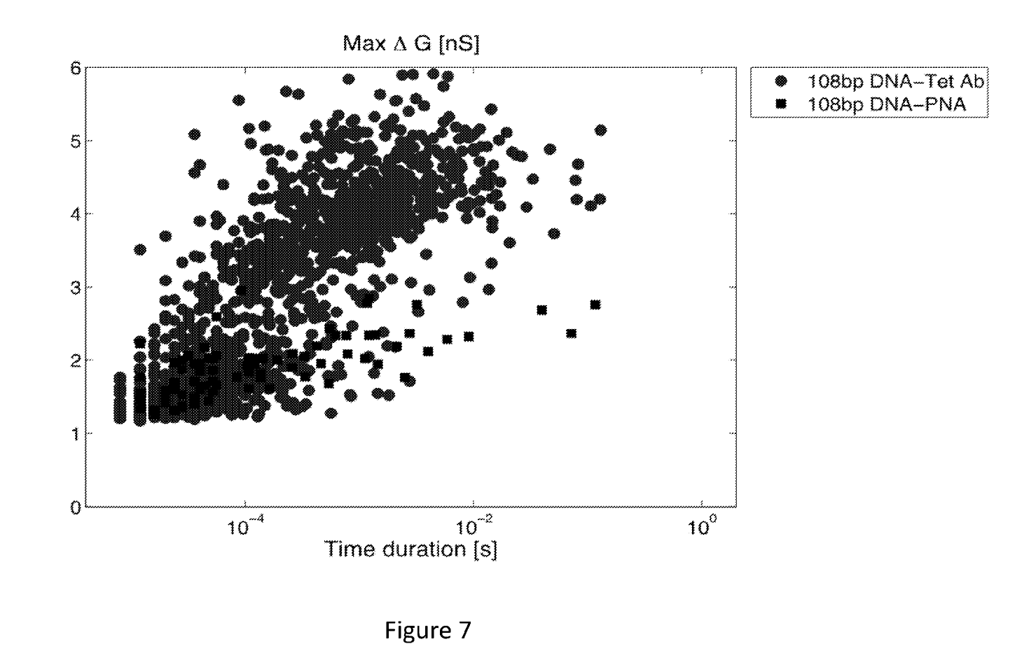

FIG. 7 shows an event plot of maximum conductance shift vs. duration of individual molecules of the Driver DNA and the Drug (antibody) passing through a pore with and estimated diameter of 27 nm and a pore thickness of 29 nm, in 1 M LiCl. Maximum conductance shift is defined as the maximum current attenuation shift divided by the applied voltage (100 mV in this case). When the driver and the drug are not coupled to each other, neither gives an appreciable signal through the nanopore and thus are minimally detected (Driver DNA signal depth was 1 to 2.5 nS, with duration 10-5 to 10-4 s, and event rate was less than 8 per minute, Drug antibody was not detected at all). However, the full complex of Driver-Drug gives 170 events per minutes and a very pronounced signal depth (2-6 nS) and duration (10-4-10-2 s) provided by 1) the length of the Driver DNA and 2) the “bulk” of the Drug Antibody. This results in DNA/Drug complexes generating an easily detectable population (dots, FIG. 7) compared to the units individually (driver DNA-PNA only black squares, FIG. 7). This shows the importance of binding between the drug and the driver molecule for nanopore detection and quantitation.

### b. Example 2

### i. Target-Linker-Driver-Payload Complex

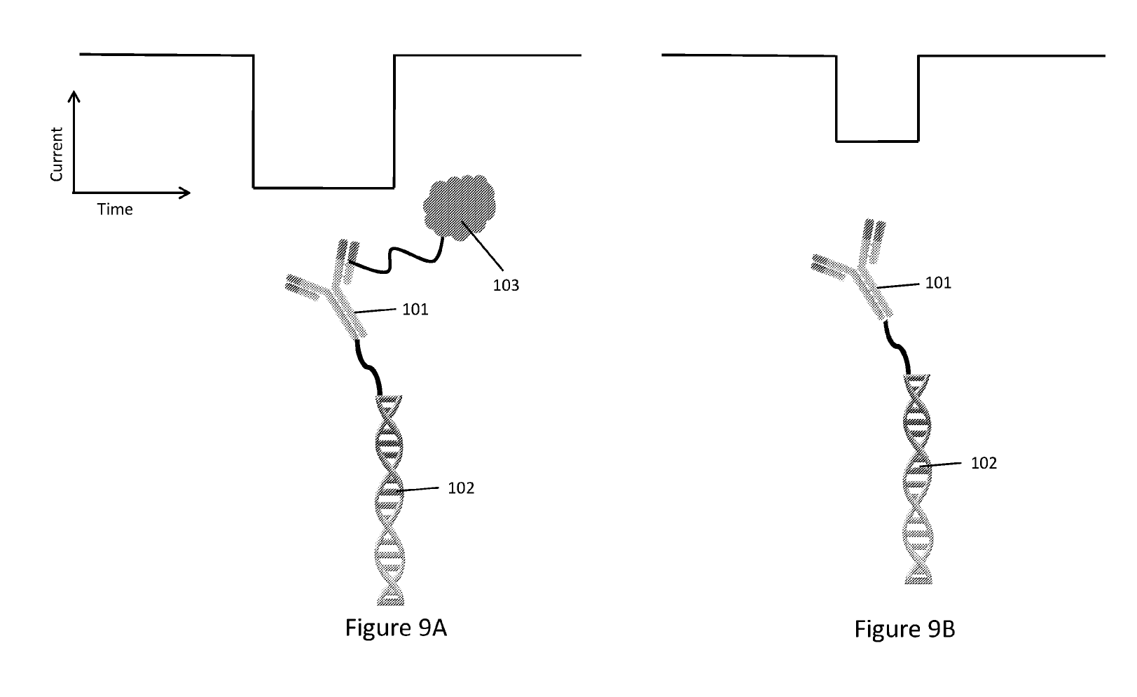

FIG. 4 provides another embodiment of the invention wherein the target molecule 101 is bound to both a driver molecule 102 via a linker molecule 104a, and a payload molecule 103 via another linker 104b. As illustrated in FIGS. 9A-B, the addition of the payload molecule enhances the electrical signal to increase its duration and/or difference from the open channel signal to facilitate detection of the target molecule in the nanopore.

### ii. Nanopore Detection of Target (Peptide Biologic)-Linker (PNA)-Driver (DNA)-Payload (Antibody) Complex

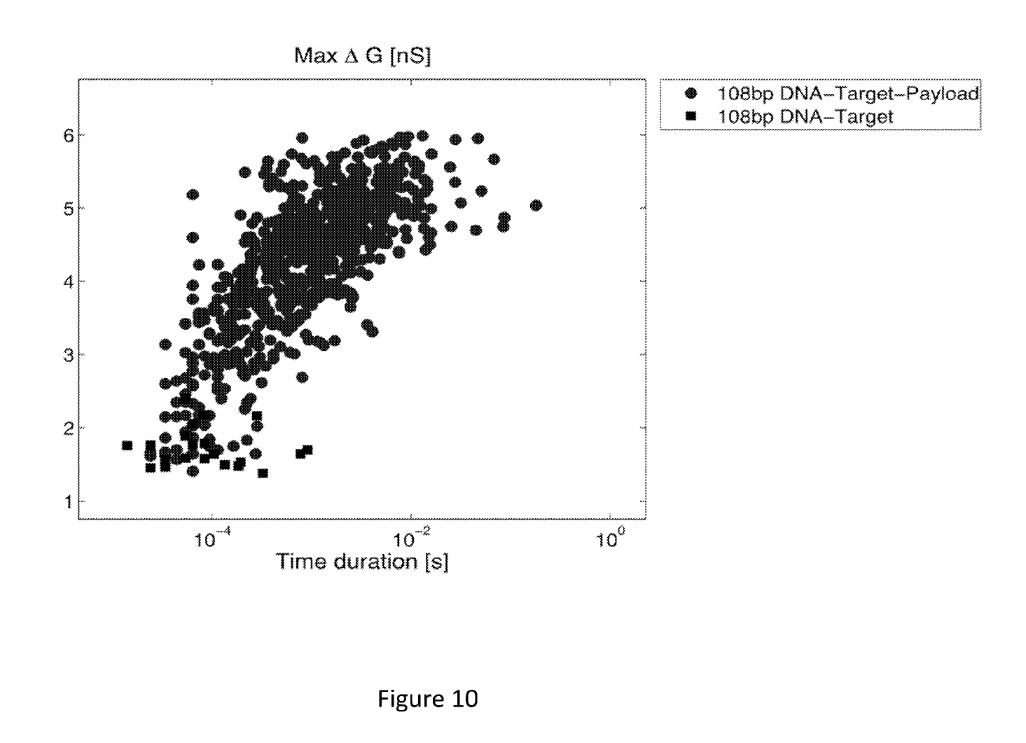

FIG. 10 provides a data collected from peptide biologic target molecule attached to a driver molecule and a payload molecule that specifically binds to the peptide target molecule. The payload molecule adds bulk to complex (driver-target-payload), thereby facilitating detection of the target molecule in the pore (as described in FIG. 9). Specifically, the peptide biologic target molecule was engineered to have a maleimide attachment site for subsequent attachment to compounds with free thiols. The engineered peptide biologic target molecule was attached to a 108 bp dsDNA driver molecule a PNA linker that binds to the driver molecule. The target molecule was further bound to an antibody that acts as the payload.

FIG. 10 shows an event plot of maximum conductance shift vs. duration of electrical signals generated by the 108 bp dsDNA driver molecule-target molecule complex as it passes through the pore. The target-driver complex without a payload molecule efficiently shuttled through the pore due to the negative charge of the DNA. However, it was not easily detectable because of its small size and short length of the driver (squares). The event population (squares) is only marginally detected (only 2 events per minute with a depth of 1 nS and duration of 10-4 s). When the DNA/Target is attached to a payload molecule (antibody), making the DNA/Peptide/Antibody complex (a.k.a. Driver/Target/Payload), the complex is easily detectable in the pore (dots). The full complex population generated an event depth of 4-6 nS and duration of 0.1 to 10 ms, and capture of 56 events per minute, significant increases compared to the driver-target complexes without the payload.

### iii. Nanopore Detection of Target (Biotin)-Driver (DNA)-Payload (Antibody) Complex

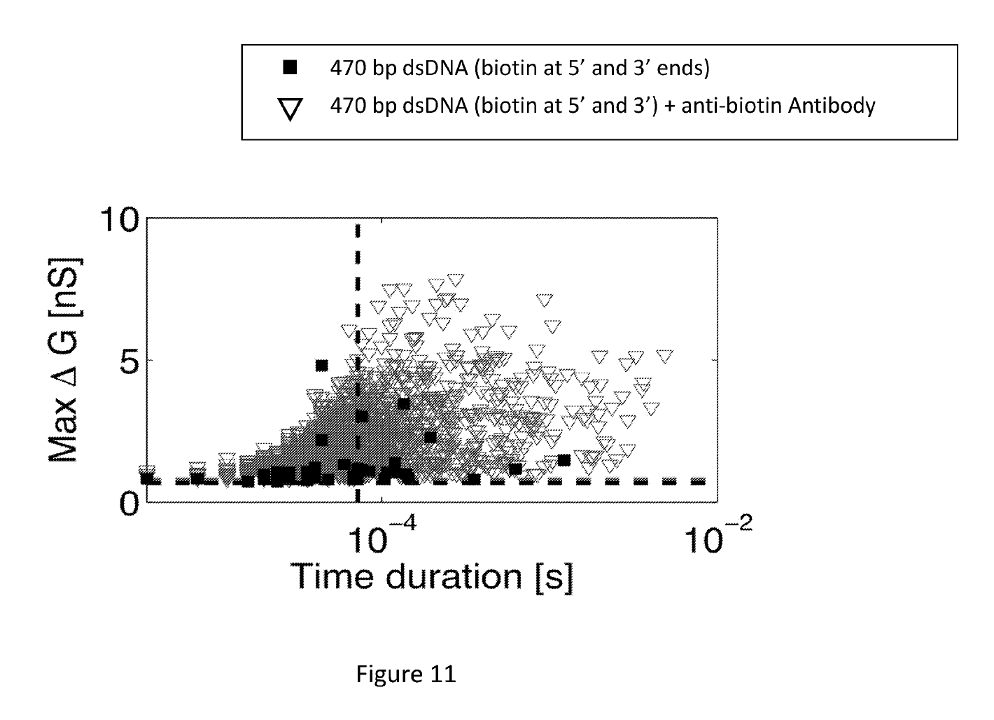

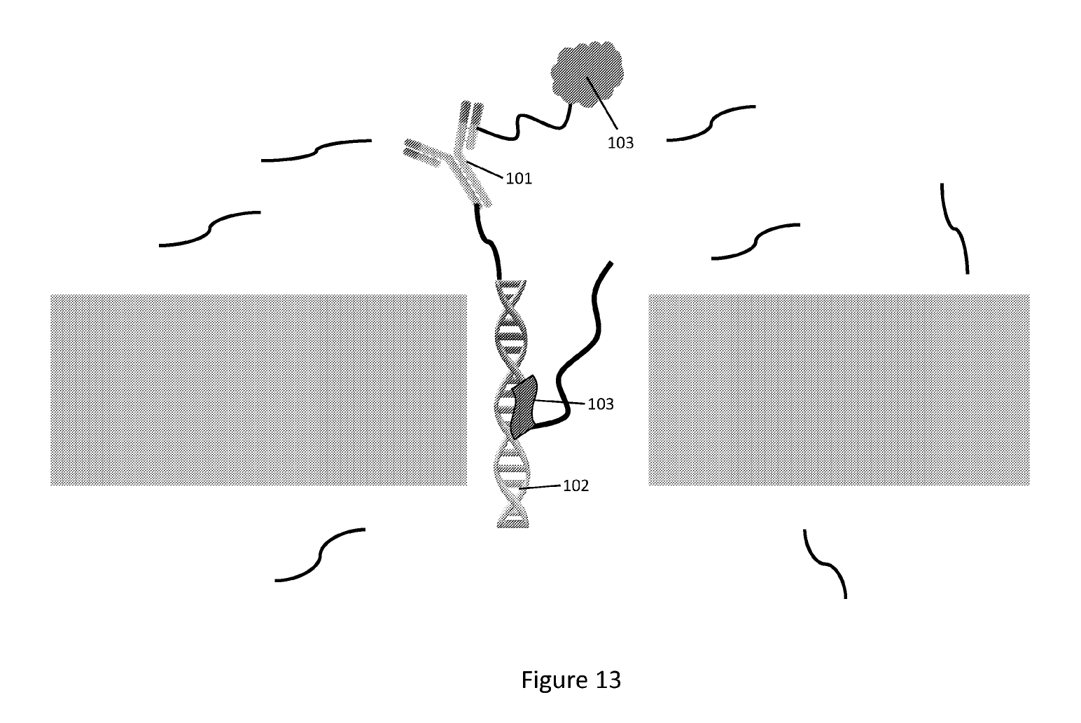

FIG. 11 provides data from detection of a target molecule when it is directly attached to a driver and a payload. Here, the 5′ and 3′ ends of DNA driver molecule was covalently attached to a small (244 Da) target molecule, biotin (vitamin B7), which was then bound by an anti-biotin antibody payload to enable detection of the target biotin molecule. For the attachment, biotin was engineered with an amide for the purpose of subsequent attachment to the 5′ phosphate on a carbohydrate. The driver molecule was 470 bp dsDNA, which was minimally detected in a nanopore (approximately 1 event detected per minute, squares in FIG. 11). The target molecule, biotin, is extremely small (244 Da), and not detected in the nanopore by itself. The nanopore signal of the driver plus biotin was much like the signal from the driver alone, thus rendering biotin undetectable. By contacting biotin-driver with the antibody, the driver-target-payload complex was formed, and the complex was easily detected. The reverse triangles in the scatter plot of FIG. 13 show events from the driver-target-payload complex which range in duration (10-4 s to 10-2 s), amplitude (1 nS to 7 nS), and frequency (1 event per second). The payload molecules by themselves do not provide sufficient signals for reliable detection. In addition, the anti-biotin antibody does not bind nonspecifically to the driver dsDNA molecule. Thus, the pronounced events are unique to the full driver-target-payload complexes, for implicit detection of the presence of the target molecule.

### c. Example 3

### i. Target-Linkers-Driver-Two Payloads Complex

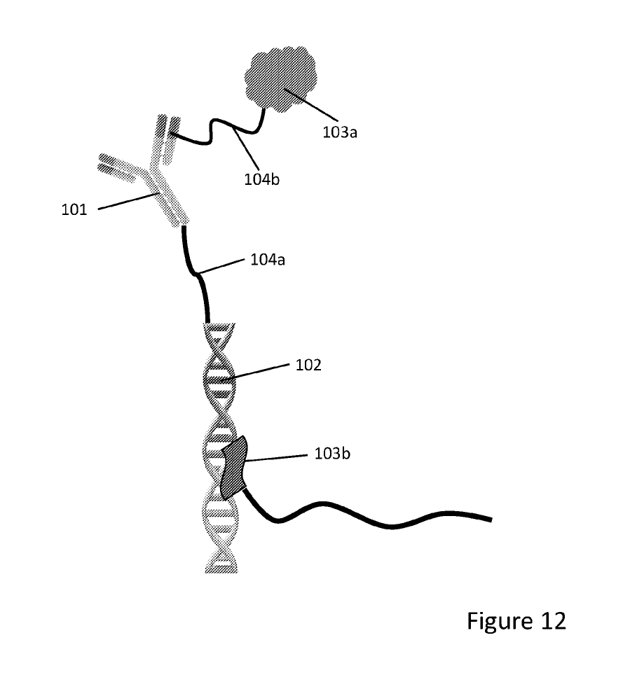

FIG. 12 provides another embodiment of the invention, wherein an additional payload molecule 103b (wavy box) is linked to the driver molecule 102. For example, a PNA-PEG payload molecule 103b can attach to a DNA driver molecule. This binding of the payload molecule further modify the electrical signal upon translocation through the nanopore to enhance detection by the nanopore. In some embodiments, the target molecule bound to the driver molecule and a plurality of payload molecules is configured to completely translocate through the nanopore from one chamber to the other (FIG. 13).

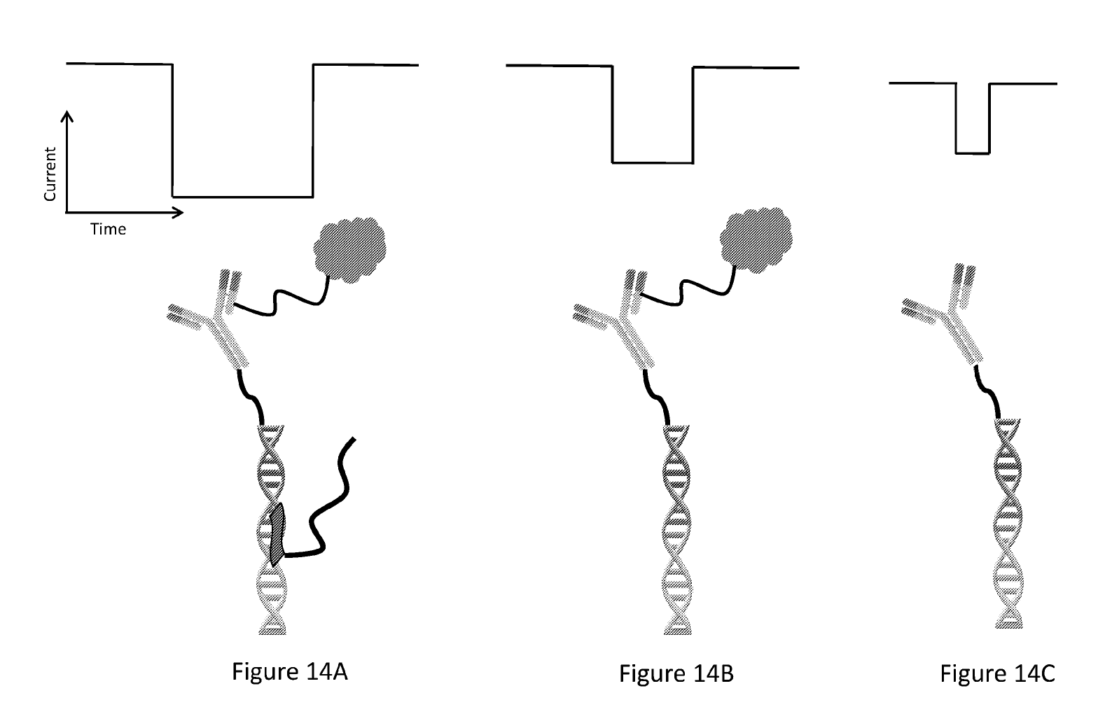

A complex comprising two payload molecules (FIG. 14A), a complex comprising one payload molecule (FIG. 14B), and a complex without any payload molecule (FIG. 14C) generate different electrical signals. The electrical signal generated by the target molecule bound to the additional payload molecule can have an enhanced duration or change in current from the open channel (FIG. 14A). The electrical signal from the additional payload molecule can also provide a current signature that is unique to facilitate discrimination over background molecules or over other target molecules.

### ii. Nanopore Detection of Target (Peptide)-Linker (PNA)-Driver (DNA)-Two Payload (Antibody as a First Payload and PNA-PEG as a Second Payload) Complex

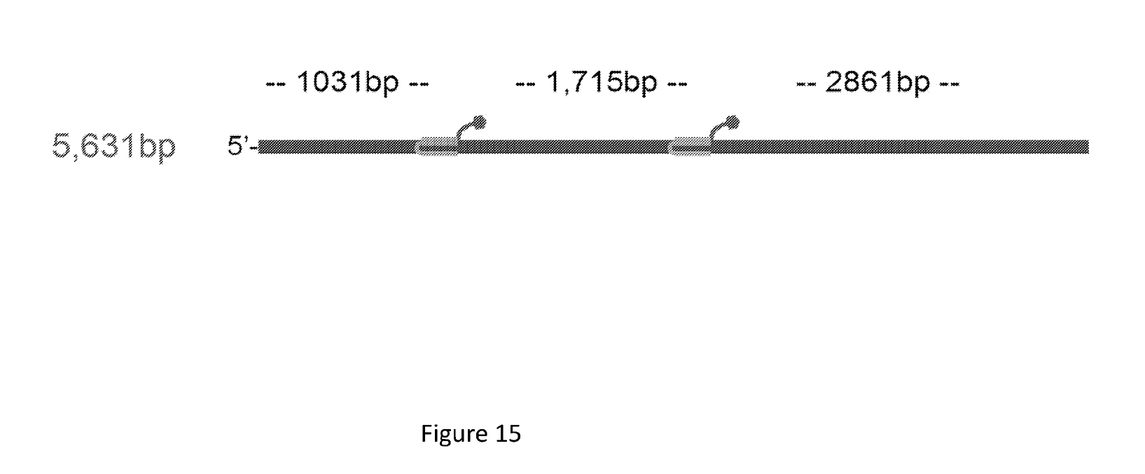

FIG. 15 shows a driver molecule used in this embodiment. The driver molecule comprises two binding sites, one binding site for a target molecule bound to a first payload molecule and the other binding site for a second payload molecule. The secondary payload attached to the driver molecule allows multiplexing. By using differently sized or spaced secondary payload, a unique impedance signature can be generated for each driver-target species in the sample.

Specifically, the two binding sites of the 5631 bp dsDNA driver molecule in FIG. 15 can bind to two PNA molecules. One binding site is for the PNA-PEG used as the secondary payload molecule for unique identification, and the other binding site is for a target-payload complex, wherein the target is the PNA-peptide and the payload is an antibody, respectively. In this model, as in prior examples, the target peptide itself is the modified “drug” that has the PNA conjugated to it as a means to be able to bind it to the driver (dsDNA) for nanopore deteciton and quantitation. The PNA used with the PEG (secondary payload) and with the peptide (target) are the same in this example, but in practice unique PNA sequences could be used and may be preferred to increase the likelihood of having only one secondary payload and one target-payload per scaffold. Since the PNA sequences were the same, the initial 10×PNA-PEG was used to bind the majority of scaffolds with only 1 of the PNA binding sites, followed by 10×PNA-peptide meant to occupy the remaining PNA binding site on the majority of scaffolds. The reagents were sequentially tested on the same 24.5-25.5 nm diameter pore.

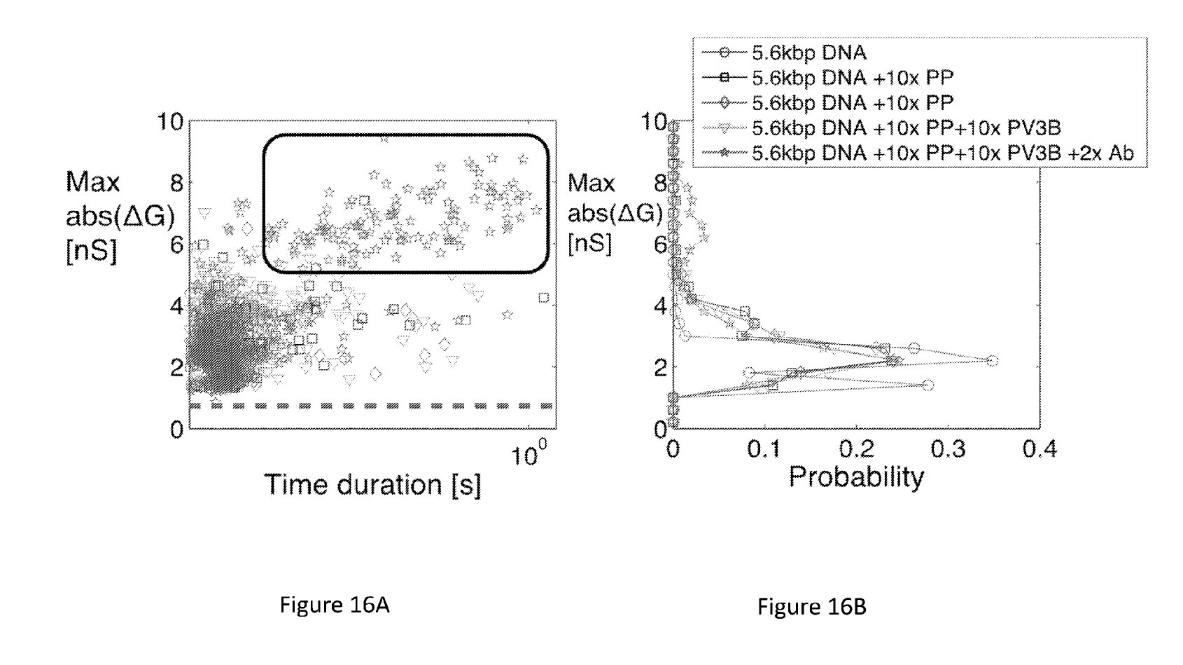

FIGS. 16A-B show the event plots (FIG. 16A) and histograms of electrical signals (FIG. 16B) collected from 0.3 nM 5631 bp DNA alone (driver alone, circles); DNA with 10×PNA-10 kDa PEG (PP) (driver-payload: squares and diamonds); DNA with 10×PP and 10×PNA-V3 loop peptide (PV3B) (driver-payload-target complex: green triangles); and DNA with 10×PP, and 10×PV3B and 2×HIV Ab to binding sites (driver-target-two payload complex: stars).

Between all reagent sets, event-free (i.e., 10 or less events) periods of buffer only were recorded for 5-10 min, to ensure each reagent tested was measured with minimal cross-sample contamination. The 5.6 kb DNA alone at 0.3 nM produced 457 events in 10 minutes, followed by 0.3 nM DNA with 10× (6 nM=2×3 nM per site) PNA-PEG (10 kDa) producing 294 events in 8 minutes, and subsequently a repeat of 0.3 nM DNA with 10×PNA-PEG (10 kDa) producing 316 events in 7 minutes. Next, DNA with 10×PP and 10× V3B produced 787 events in 15 minutes, followed by DNA with 10×PP and 10× V3B and 2× Ab which produced 828 events in 25 minutes. As a final control, 2× Ab alone (0.6 nM) was run, producing only 7 events in 10 minutes.

The DNA alone population is distinguishable from the others, producing the standard folded, partially folded and unfolded event profiles. The DNA with 10×PP and PV3B populations are comparable in event distributions, while the full complex with Ab present (as in the payload free case) produces a deeper and longer lasting population (FIG. 16A, box around events, left). These aggregate trends using the standard event plots shows that complexes with secondary payload binding and with the target-payload binding produce a unique electrical signature for target detection and quantitation.

Alternative ways of designing driver molecules are available for this embodiment. For example, PCT Publication WO/2015/176034, incorporated by reference in its entirety, discloses a driver molecule (i.e., scaffold) comprising multiple binding sites for unique identifiers and targets, as means of multiplexing. Such driver molecules can be linked to a target molecule to enable detection of the target molecule using a nanopore device.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A complex designed for nanopore detection, comprising:
a target molecule comprising a first attachment site; and
a driver molecule comprising a first attachment site,
wherein the first attachment site of the driver molecule makes a covalent bond to the first attachment site of the target molecule.

2. A complex designed for nanopore detection, comprising:
a target molecule comprising a first attachment site; and
a linker molecule comprising a first attachment site and a second attachment site,
wherein the first attachment site of the linker molecule makes a covalent bond to the first attachment site of the target molecule, and
the second attachment site of the linker molecule is a binding site for a driver molecule.

3. The complex of claim 2, further comprising the driver molecule,
wherein the driver molecule is bound to the second attachment site of the linker molecule via a covalent bond, an intermediate linker, a Van der Waals bond, an electrostatic bond, a hydrophobic interaction, a pi-stacking interaction, an ionic bond, or another non-covalent electrostatic interaction.

4. The complex of claim 2, wherein the linker molecule comprises a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid (PNA), a polyethylene glycol (PEG), a dendrimer, a synthetic polymer, or any combination thereof.

5. The complex of claim 2, wherein the linker molecule comprises a peptide, a valine-citrulline linker, a maleimidocaproyl (mc) linker, an [N-maleimidomethyl]cyclohexane-1-carboxylate (MCC) linker, a succinimidyl 4-[N-maleimidomethyl]cyclohexane-1-carboxylate (SMCC) linker, a PEG-based linker, a hydrazone linker, an N-succinimidyl-4-(2-pyridyldithio) butanoate (SPDB) linker, or a carbonate linker, or any other homobifunctional or heterobifunctional molecule.

6. The complex of claim 1, wherein the target molecule comprises a biological therapeutic.

7. The complex of claim 6, wherein the biological therapeutic is a monoclonal antibody, a protein, a protease, a peptide, a sugar, a nucleic acid, or any combination thereof.

8. The complex of claim 1, wherein the target molecule comprises a small molecule.

9. The complex of claim 8, wherein the small molecule is a small molecule therapeutic or small molecule diagnostic drug.

10. The complex of claim 9, wherein the small molecule is a small molecule therapeutic drug selected from the group consisting of an enzyme inhibitor or activator, a receptor antagonist or agonist, and a small molecule modifier of a cellular process or pathway.

11. The complex of claim 10, wherein the small molecule therapeutic drug is Lisinopril.

12. The complex of claim 1, wherein the target molecule comprises an ingredient of a chemical or biological product, selected from the group consisting of a fertilizer, a construction or building material, and a dietary composition.

13. The complex of claim 12, wherein the ingredient is a nitrogenase.

14. The complex of claim 12, wherein the ingredient is boronic acid.

15. The complex of claim 1, wherein the target molecule is an indicator selected from the group consisting of pH indicator, a toxin indicator, a chemical indicator, a pollutant indicator, or a temperature indicator.

16. The complex of claim 1, wherein the driver molecule is a single stranded deoxyribonucleic acid (ssDNA), a double stranded deoxyribonucleic acid (dsDNA), or a ribonucleic acid (RNA).

17. The complex of claim 16, wherein the driver molecule comprises more than 20 nucleotides.

18. The complex of claim 16, wherein the driver molecule binds to the linker molecule via a covalent bond, an intermediate linker, a Van der Waals bond, an electrostatic bond, a hydrophobic interaction, a pi-stacking interaction, an ionic bond, or another non-covalent electrostatic interaction.

19. The complex of claim 1, wherein said target molecule further comprises a second attachment site, wherein said second attachment site is bound to a first payload molecule.

20. The complex of claim 19, wherein the first payload molecule is bound to the target molecule via a covalent bond, an intermediate linker, a Van der Waals bond, an electrostatic bond, a hydrophobic interaction, a pi-stacking interaction, an ionic bond, or another non-covalent electrostatic interaction.

21. The complex of claim 1, wherein said driver molecule further comprises a second attachment site, wherein said second attachment site is bound to a second payload molecule.

22. The complex of claim 21, wherein the second payload molecule is bound to the driver molecule via a covalent bond, an intermediate linker, a Van der Waals bond, an electrostatic bond, a hydrophobic interaction, a pi-stacking interaction, an ionic bond, or another non-covalent electrostatic interaction.

23. The complex of claim 19, wherein the first or the second payload molecule is a peptide, a protein, a carbohydrate, a single stranded deoxyribonucleic acid (ssDNA), a double stranded deoxyribonucleic acid (dsDNA), a ribonucleic acid (RNA), a nanoparticle, a peptide nucleic acid, a polyethylene glycol (PEG), a dendrimer, or any combination thereof.

24. The complex of claim 19, wherein the target molecule is bound to a plurality of payload molecules.

25. The complex of claim 19, wherein the driver molecule is bound to a plurality of payload molecules.

26. The complex of claim 1, wherein the first attachment site of the target molecule comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

27. The complex of claim 1, wherein the first attachment site of the driver molecule comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

28. A method of detecting a target molecule, comprising the steps of:
a. loading a sample suspected to comprise the complex of claim 1 in a nanopore device comprising at least one nanopore;
b. applying a voltage across said at least one nanopore; and
c. measuring an electrical signal through the pore that correlates with the presence or absence of the complex in the sample.

29. The method of claim 28, wherein said sample comprises a pharmaceutical composition.

30. The method of claim 28, wherein said sample comprises a blood,
urine, saliva, or tissue sample from a patient.

31. The method of claim 28, wherein said sample comprises water, soil, air, sludge, petroleum, or a chemical or biological product.

32. The method of claim 28, wherein the complex is bound to one or more payload molecules, and wherein the electrical signal further correlates with the binding of said one or more payload molecules.

33. The method of claim 28, further comprising a step of determining the presence or absence of the complex in the sample based on the electrical signal.

34. The method of claim 28, wherein the electrical signal further correlates with the presence or absence of the target molecule in the sample.

35. The method of claim 34, further comprising a step of determining the presence or absence of the target molecule in the sample based on the electrical signal.

36. The method of claim 28, further comprising the step of determining the concentration of the complex or the target molecule in the sample.

37. A modified target molecule designed for nanopore detection, comprising
a first attachment site for a driver molecule or a linker molecule, wherein the attachment site is generated by a first modification of a target molecule.

38. The modified target molecule of claim 37, wherein the first modification comprises biotinylation, acetylation, methylation, summolation, glycosylation, phosphorylation, or oxidation.

39. The modified target molecule of claim 37, wherein the first modification comprises chemical or biological synthesis.

40. The modified target molecule of claim 37, wherein the first modification comprises a genetic engineering of the target.

41. The modified target molecule of claim 37, wherein the first attachment site comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

42. The modified target molecule of claim 37, wherein the first attachment site can bind to the driver molecule via a covalent bond, an intermediate linker, a Van der Waals bond, an electrostatic bond, a hydrophobic interaction, a pi-stacking interaction, an ionic bond, or another non-covalent electrostatic interaction.

43. The modified target molecule of claim 37, wherein the first attachment site can bind to the linker molecule via a covalent bond, an intermediate linker, a Van der Waals bond, an electrostatic bond, a hydrophobic interaction, a pi-stacking interaction, an ionic bond, or another non-covalent electrostatic interaction.

44. The modified target molecule of claim 37, wherein the target molecule comprises a biologic therapeutic.

45. The modified target molecule of claim 44, wherein the biologic therapeutic is a monoclonal antibody, a protein, a protease, a peptide, a sugar, a nucleic acid, or any combination thereof.

46. The modified target molecule of claim 37, wherein the target molecule comprises a small molecule.

47. The modified target molecule of claim 46, wherein the small molecule is a small molecule therapeutic or small molecule diagnostic drug.

48. The modified target molecule of any of claim 47, wherein the small molecule is a small molecule therapeutic selected from the group consisting of an enzyme inhibitor or activator, a receptor antagonist or agonist, and a small molecule modifier of a cellular process or pathway.

49. The modified target molecule of claim 48, wherein the small molecule therapeutic drug is Lisinopril.

50. The modified target molecule of claim 37, wherein the target molecule is an ingredient of a chemical or biological product selected from the group consisting of a fertilizer, a construction or building material, and a dietary composition.

51. The modified target molecule of claim 50, wherein the ingredient is a nitrogenase.

52. The modified target molecule of claim 50, wherein the ingredient is boronic acid.

53. The modified target molecule of claim 37, wherein the target molecule is selected from the group consisting of pH indicator, a toxin indicator, a chemical indicator, a pollutant indicator, or a temperature indicator.

54. The modified target molecule of claim 37, wherein the modified target molecule maintains at least one function of the target molecule.

55. The modified target molecule of claim 54, wherein the at least one function is therapeutic, diagnostic, biological, chemical, or nutritional activity.

56. The modified target molecule of claim 37, wherein the driver molecule is a single stranded deoxyribonucleic acid (ssDNA), a double stranded deoxyribonucleic acid (dsDNA), or a ribonucleic acid (RNA).

57. The complex of claim 56, wherein the driver molecule comprises more than 20 nucleotides.

58. The modified target molecule of claim 36, further comprising a second attachment site for binding to a payload molecule.

59. The modified target molecule of claim 58, wherein the second attachment site is generated by a second modification of the target molecule or the modified target molecule.

60. The modified target molecule of claim 59, wherein the second modification comprises biotinylation, acetylation, methylation, summolation, glycosylation, phosphorylation, or oxidation.

61. The modified target molecule of claim 59, wherein the second modification comprises chemical or biological synthesis.

62. The modified target molecule of claim 59, wherein the second modification comprises a genetic engineering of the target.

63. The modified target molecule of claim 58, wherein the second attachment site comprises a reactive group on: an amino acid, a deoxyribose nucleic acid (DNA), or a ribonucleic acid (RNA), an amine group, a thiol, an aldehyde, a ketone, an azide, an alkyne, a sulfur, a phosphorous or other reactive atom, a ketone, a carboxylic acid, an ether, an amide, alkyl halide, an ester, an alkyne, an hydroxyl, or an alcohol.

64. The modified target molecule of claim 58, wherein the second attachment site can bind to the payload molecule via a covalent bond, an intermediate linker, a Van der Waals bond, an electrostatic bond, a hydrophobic interaction, a pi-stacking interaction, an ionic bond, or another non-covalent electrostatic interaction.

65. A method of detecting the modified target molecule, comprising the steps of:
a. obtaining a sample suspected to comprise the modified target molecule of claim 37;
b. adding the driver molecule to the sample to generate a reaction product;
c. applying the reaction product to a nanopore device comprising at least one nanpore;
d. applying a voltage across said at least one nanopore; and
e. measuring an electrical signal through the pore that correlates with the presence or absence of the modified target molecule in the sample.

66. The method of claim 65, wherein said sample comprises a pharmaceutical composition.

67. The method of claim 65, wherein said sample comprises a blood, urine, saliva, or tissue sample from a patient.

68. The method of claim 65, wherein said sample comprises water, soil, air, sludge, petroleum, or a chemical or biological product.

69. The method of claim 65, wherein the modified target molecule is bound to one or more payload molecules, and wherein the electrical signal further correlates with the binding of said one or more payload molecules.

70. The method of claim 65, further comprising the step of determining the presence or absence of the modified target molecule in the sample based on the electrical signal.

71. The method of claim 70, further comprising the step of determining the concentration of the modified target molecule in the sample.
